(19) United States
(12) Patent Application Publication
(10) Pub. No.: US 2016/0030535 A1
(43) Pub. Date: Feb. 4, 2016

(54) Method of Induction and Purification of a Cell Population Responsible for Vascular Mimicry and Use of Same

(71) Applicant: NEOSTEM ONCOLOGY, LLC, Irvine, CA (US)

(72) Inventors: Aleksandra J. Poole, Irvine, CA (US); Candace Y. Hsieh, Irvine, CA (US); Andrew N. Cornforth, Irvine, CA (US)

(21) Appl. No.: 14/774,538

(22) PCT Filed: Mar. 10, 2014
(86) PCT No.: PCT/US2014/022488

§ 371 Date: Sep. 10, 2015

Related U.S. Application Data
(60) Provisional application No. 61/776,654, filed on Mar. 11, 2013.

(51) Int. Cl. A61K 39/00; A61K 39/39

(52) U.S. Cl. CPC A61K 39/0011; A61K 39/39; A61K 2039/5154; A61K 2039/55522

(57) ABSTRACT

The disclosure provides cancer stem cells responsible for vascular mimicry, for use in stimulating immune response against a cancer. Methods for preparing and purifying the cancer stem cells are provided.

### CROSS-REFERENCE TO RELATED APPLICATIONS

The present application claims the benefit under 35 U.S.C. §119(e) to U.S. Provisional Patent Application 61/776,654 filed Mar. 11, 2013, the entire content of which are incorporated by reference herein.

### FIELD OF THE DISCLOSURE

The disclosure concerns cell lines that can induce angiogenesis, where the cell line is obtained from a malignant tumor of a cancer patient.

### BACKGROUND OF THE DISCLOSURE

Aggressive cancers have the ability to sequester blood flow and acquire their own nourishment by way of angiogenesis or vasculogenesis. A number of anti-angiogenic drugs are available, including those that target endothelial cells, including bevacizumab, marketed as AVASTIN® (Genentech). However, these drugs have given disappointing results, in terms of endpoints such as objective response and clinical outcome.

New vasculogenic-like networks are formed in tumors by tumor cells themselves, rather than by epithelial cells in a process referred to as vasculogenic mimicry, or vascular mimicry (VM), due to tumor cells mimicking epithelial cells in the process of vasculogenesis. Vascular mimicry has been characterized in human uveal melanoma in which tumor cells formed channels, microcirculatory networks and tubular structures composed of extracellular matrix lined with tumor cells. These pathways allowed for nourishment of rapidly growing tumors as well as routes for metastases to escape.

Since its original characterization in uveal melanoma, VM has been shown to occur in other cancers, including cutaneous melanoma, breast, ovarian, prostate, and glioblastoma multiforme. Highly invasive, but not poorly invasive, melanoma cells exhibit a genetic reversion to a pluripotent embryonic-like genotype. The plasticity of these pluripotent cancer cells allows them to mimic the activities of endothelial cells and contribute to VM in a way similar to the way that cancer stem cells (CSC) possess a pluripotent embryonic-like phenotype and possess the capacity to differentiate into the parental cancer cells.

### SUMMARY

The present disclosure provides vascular mimicry (VM) cancer stem cells (CSC) for use in in autologous immune therapy against neoplasms. In general, the majority of the tumor cells are fairly differentiated and mixed with normal cells such as blood vessel constituents, connective tissue and normal host tissue. Therefore when the bulk tumor is used, the immune response is directed against the more differentiated cells allowing the cancer stem cells to elude the attack and the possibility to relapse or metastases the tumor. Isolating and culturing pure populations of the cancer stem cells responsible for VM can provide a source of antigen for immunotherapy, as well as, be a model system for anti-angiogenic drug discovery and diagnosing cancer severity.

Also provided is a method for preparing a mixture of tumor antigens originating from a tumor sample or biopsy from a donor, comprising the steps of: (a) dissociating at least a portion of the tumor cells from the tumor sample or biopsy; and (b) isolating a population of vascular mimicry cancer stem cells.

Thus, disclosed herein is an immunogenic composition comprising dendritic cells activated ex vivo by tumor antigens derived from the population of purified vascular mimicry (VM) cancer stem cells (CSC). In certain embodiments, the tumor antigens comprise cell extracts of the VM-CSC, lysates of the VM-CSC, or intact VM-CSC cells. In another embodiment, the tumor antigens comprise messenger RNA transfected into the dendritic cells ex vivo.

In another embodiment, the intact VM-CSC are rendered non-proliferative. In another embodiment, the intact VM-CSC are rendered non-proliferative by irradiation. In another embodiment, the intact VM-CSC are rendered non-proliferative by exposure of the cells to a nuclear or protein cross-linking agent.

In another embodiment, the immunogenic composition further comprises a pharmaceutically acceptable carrier and/or excipient. In another embodiment, the immunogenic composition further comprises an adjuvant. In another embodiment, the adjuvant is granulocyte macrophage colony stimulating factor.

In another embodiment, the immunogenic composition comprises activated dendritic cells and VM-CSC.

Also provided is a method of treating cancer in a subject in need thereof, comprising administration of an immunogenic composition disclosed herein to the subject. In another embodiment, the cancer is adrenocortical carcinoma, anal cancer, appendix cancer, astrocytoma, basal-cell carcinoma, bile duct cancer, bladder cancer, bone cancer, brain cancer, breast cancer, bronchial adenomas/carcinoids, carcinoid tumor, cervical cancer, chronic myeloproliferative disorders, colon cancer, desmoplastic small round cell tumor, endometrial cancer, ependymoma, esophageal cancer, Ewing's sarcoma, germ cell tumors, eye cancer, gallbladder cancer, gastric cancer, gastrointestinal carcinoid tumor, gastrointestinal stromal tumor (GIST), gestational trophoblastic tumor, glioma, gastric carcinoid, head and neck cancer, heart cancer, hepatocellular cancer, Hodgkin lymphoma, hypopharyngeal cancer, islet cell carcinoma, Kaposi sarcoma, kidney cancer, leukemias, lip and oral cavity cancer, liposarcoma, liver cancer, lung cancer, lymphomas, macroglobulinemia, medulloblastoma, melanoma, merkel cell carcinoma, mesothelioma, mouth cancer, multiple myeloma/plasma cell neoplasm, mycosis fungoides, nasal cavity and paranasal sinus cancer, nasopharyngeal carcinoma, neuroblastoma, oral cancer, oropharyngeal cancer, ovarian cancer, ovarian epithelial cancer, pancreatic cancer, parathyroid cancer, penile cancer, pharyngeal cancer, pheochromocytoma, pineal astrocytoma, pineal germinoma, pineoblastoma, pituitary adenoma, pleuropulmonary blastoma, prostate cancer, rectal cancer, renal cell carcinoma, retinoblastoma, rhabdomyosarcoma, salivary gland cancer, sarcoma, Sézary syndrome, skin cancer, squamous cell carcinoma, stomach cancer, testicular cancer, throat cancer, thymoma, thyroid cancer, urethral cancer, uterine cancer, uterine sarcoma, vaginal cancer, vulvar cancer, Waldenström macroglobulinemia, or Wilms tumor.

In one embodiment, the immunogenic composition is administered in a plurality of doses, each dose comprising about 5-20×10⁶ cells. In another embodiment, the dose comprises about 10×10⁶ cells. In another embodiment, the dose is administered weekly for 2-5 doses, followed by monthly for 3-6 doses. In yet another embodiment, the subject receives from 6-10 doses of immunogenic composition.

Also provided is the use of an immunogenic composition disclosed herein in the manufacture of a medicament for the treatment of cancer.

Also provided is the use of an immunogenic composition disclosed herein for the treatment of cancer.

Further provided herein is a method for preparing a population of vascular mimicry (VM) cancer stem cells (CSC), the method comprising: acquiring a sample of a tumor; dissociating the cells of the sample, in vitro culturing the dissociated cells in a defined medium on a non-adherent substrate, wherein the defined medium is serum free and is supplemented with at least one growth factor that acts through the mitogen activated protein kinase (MAPK) pathway, thereby forming CSC spheroids; optionally in vitro culturing the CSC-spheroids to form early CSC, mixed CSC, or epithelial to mesenchymal transitioned (EMT)-CSC; and culturing the CSC spheroids, the early CSC, mixed CSC, or EMT-CSC in a defined medium on an adherent substrate, wherein the defined medium contains a serum source and is supplemented with a soluble laminin, thereby forming a population of VM-CSC, wherein at least 80% of the cells in the VM-CSC population express two or more of the biomarkers VEGF-R2, VE-cadherin, VEGF-A, CD34, vWF, and PECAM. In another embodiment, the defined medium in any step further comprises at least one receptor tyrosine kinase (RTK) ligand. In another embodiment, at least 80% of the cells in the VM-CSC population further express one or more of the biomarkers VEGF-R1 and UEA-1. In yet another embodiment, at least 90% of the cells in the VM-CSC population express two or more of the biomarkers VEGF-R2, VE-cadherin, VEGF-A, CD34, vWF, and PECAM. In another embodiment, at least 80% of the cells in the CSC spheroid population express two or more of the biomarkers EpCAM, CD117, ALDH, CD133, CD24, Ki-67.

In one embodiment, the soluble laminin comprises an alpha1, alpha2, alpha3, or alpha4 chain. In another embodiment, the laminin is in a monomer, dimer, or trimer form. In yet another embodiment, the laminin is not an insoluble polymer form.

In another embodiment, at least 80% of the cells in the CSC spheroid population further express one or more of the biomarkers NCAM, vimentin, CK8, TGFβR, EGFR, CD44, ABCG2, Slug/Snail, nestin, and TP53. In another embodiment, at least 90% of the cells in the CSC spheroid population express two or more of the biomarkers EpCAM, CD117, ALDH, CD133, CD24, Ki-67.

In yet another embodiment, the early CSC are generated by culturing the CSC spheroids in a defined medium on an adherent substrate, wherein the defined medium is serum free and is supplemented with at least one growth factor that acts through the MAPK pathway, thereby forming a population of early CSC, wherein at least 80% of the cells in the early CSC population express two or more of the biomarkers EpCAM, CD133, CD44, Nanog, Sox2, Oct3/4, CD17, and Ki-67. In another embodiment, at least 80% of the cells in the early CSC population further express one or more of the biomarkers TGFβR and CD24. In another embodiment, at least 90% of the cells in the early CSC population express two or more of the biomarkers EpCAM, CD133, CD44, Nanog, Sox2, Oct3/4, CD17, and Ki-67.

In yet another embodiment, the mixed CSC are generated by culturing the CSC spheroids in a defined medium on an adherent substrate, wherein the defined medium contains serum, thereby forming a population of mixed CSC, wherein at least 80% of the cells in the mixed CSC population express two or more of the biomarkers ABCG2, CD133, CD24, CD44, CD34, CD117, CK8, EpCAM, Ki-67, Nanog, N-cadherin, NCAM, Oct3/4, Slug/Snail, Twist, vimentin, ALDH, TGFβR, Sox2, EGFR) nestin, TP53, VEGF-R1, VEGF-R2, VE-cadherin, VEGF-A, vWF, PECAM, and UEA-1. In another embodiment, the defined medium further comprises at least one receptor tyrosine kinase (RTK) ligand. In another embodiment, at least 90% of the cells in the mixed CSC population express two or more of the biomarkers ABCG2, CD133, CD24, CD44, CD34, CD117, CK8, EpCAM, Ki-67, Nanog, N-cadherin, NCAM, Oct3/4, Slug/Snail, Twist, vimentin, ALDH, TGFβR, Sox2, EGFR) nestin, TP53, VEGF-R1, VEGF-R2, VE-cadherin, VEGF-A, vWF, PECAM, and UEA-1.

In yet another embodiment, the EMT-CSC are generated by culturing the CSC spheroids in a defined medium on an adherent substrate, wherein the defined medium contains serum and is supplemented with at least one growth factor that acts through the MAPK pathway, thereby forming a population of EMT-CSC, wherein at least 80% of the cells in the EMT-CSC population express two or more of the biomarkers NCAM, Slug/Snail, CD24, and Twist. In another embodiment, at least 80% of the cells in the EMT-CSC population further express one or more of the biomarkers CD133, Nanog, CD117, N-cadherin, CD44, and vimentin. In yet another embodiment, at least 90% of the cells in the EMT-CSC population express two or more of the biomarkers NCAM, Slug/Snail, CD24, and Twist.

Also provided herein is a method for preparing a population of vascular mimicry (VM) cancer stem cells (CSC), the method comprising: acquiring a sample of a tumor; dissociating the cells of the sample, in vitro culturing the dissociated cells in a defined medium on a non-adherent substrate, wherein the defined medium is serum free and is supplemented with at least one growth factor that acts through the MAPK pathway, thereby forming CSC spheroids; in vitro culturing the CSC spheroids to form early CSC, mixed CSC, or EMT-CSC; and in vitro culturing the early CSC, mixed CSC, or EMT-CSC in a defined medium on an adherent substrate, wherein the wherein the defined medium is supplemented with a soluble laminin, thereby forming VM-CSC; wherein the at least 80% of the cells in the VM-CSC population express two or more of the biomarkers VEGF-R2, VE-cadherin, VEGF-A, CD34, vWF, and PECAM. In another embodiment, the defined medium in any of the steps further comprises at least one RTK ligand. In another embodiment, at least 80% of the cells in the VM-CSC population further express one or more of the biomarkers VEGF-R1 and UEA-1. In yet another embodiment, at least 90% of the cells in the VM-CSC population express two or more of the biomarkers VEGF-R2, VE-cadherin, VEGF-A, CD34, vWF, and PECAM.

In one embodiment, the soluble laminin comprises an alpha1, alpha2, alpha3, or alpha4 chain. In another embodiment, the laminin is in a monomer, dimer, or trimer form. In yet another embodiment, the laminin is not an insoluble polymer form.

Also disclosed herein are methods of preparing a population of VM-CSC wherein the defined media is any media described in Table 2, any media from a combination of Table 2 and Table 3, any media from a combination of Table 2, Table 3, and Table 4, or any media from a combination of Table 2 and Table 4. In another embodiment, the growth factor is one or more of fibroblast growth factor (FGF), epidermal growth factor (EGF), or activin A. In another embodiment, the FGF is basic FGF (bFGF). In yet another embodiment, the defined medium is not supplemented with activin A. In another embodiment, the defined medium is supplemented with an agonist of activin A, in an amount effective to prevent spontaneous differentiation of CSC. In another embodiment, the media is supplemented with an antagonist of activin A, and the antagonist is follistatin or an antibody that specifically binds to activin A. In another embodiment, the defined medium in any of the steps further comprises at least one RTK ligand.

In another embodiment, the medium is not supplemented with an antioxidant. In another embodiment, the antioxidant is superoxide dismutase, catalase, glutathione, putrescine, or β-mercaptoethanol. In yet another embodiment, the medium is supplemented with glutathione.

In another embodiment, the adherent substrate is configured to adhere to, and optionally to collect, anchorage dependent cells, such as fibroblasts. In another embodiment, the non-adherent substrate is an ultralow adherent polystyrene surface. In yet another embodiment, the adherent substrate comprises a surface coated with a protein rich in RGD tripeptide motifs.

Also provided is a population of purified VM-CSC cells prepared by a method disclosed herein.

Also provided is a VM-CSC cell line prepared by a method disclosed herein.

Also provided is a method of stimulating an immune response against cancer in a subject in need thereof, comprising administration of an immunogenic composition, VM-CSC cells, or a VM-CSC cell line disclosed herein.

Also provided is the use of VM-CSC cells or a VM-CSC cell line disclosed herein in the manufacture of a medicament for the treatment of cancer.

Also provided is the use of VM-CSC cells or a VM-CSC cell line for the treatment of cancer.

### BRIEF DESCRIPTION OF THE DRAWINGS

FIG. 1 is a flow chart of the process of isolation, expansion, and harvest of cancer stem cells (CSC) from an excised tumor following by the induction of the vascular mimicry (VM) phenotype.

FIG. 2A-B. FIG. 2A depicts a commercially available melanoma cancer cell line that was exposed to conditions permissive for VM induction. Vascular tube mimicking strings of cells are extending over a thick layer of MATRIGEL® (BD Biosciences). FIG. 2B depicts a patient-isolated melanoma CSC cell line that was exposed to conditions permissive for VM induction. Extensive capillary mimicking strings of cells are extending over a thick layer of MATRIGEL®.

FIG. 3 depicts VM morphology of a CSC culture that is manifested in the shape of “buds”.

FIG. 4 depicts VM morphology of a CSC culture that is manifested in the shape of “strings” or “tubes”.

FIG. 5 depicts CSC that are expanding in a monolayer, not specific for a VM morphology.

FIG. 6A-D. FIG. 6A depicts VM-CSC vascular buds positive for the VEGF-R2 and VEGF-R1. FIG. 6B depicts a red channel image of the cells in FIG. 6A showing cells positive for VEGF-R2. FIG. 6C depicts a green channel image of the cells in FIG. 6A showing cells positive for VEGF-R1. FIG. 6D depicts a blue channel image of the cells in FIG. 6A representing the nuclear stain bisbenzimide.

FIG. 7A-D. FIG. 7A depicts VM=CSC vascular buds positive for VEGF-A and VE-cadherin. FIG. 7B depicts a red channel image of the cells in FIG. 7A showing cells positive for VEGF-A. FIG. 7C depicts a green channel image of the cells in FIG. 7A showing cells positive for VE-cadherin. FIG. 7D depicts a blue channel image of the FIG. 7A representing the nuclear stain bisbenzimide.

FIG. 8A-D. FIG. 8A depicts CSC vascular buds positive for von Willebrand Factor (vWF) and CD34. FIG. 8B depicts a red channel image of the cells of FIG. 8A showing cells positive for vWF. FIG. 8C depicts a green channel image of the cells of FIG. 8A showing cells positive for CD34. FIG. 8D depicts a blue channel image of the cells of FIG. 8.A representing the nuclear stain bisbenzimide.

FIG. 9A-D. FIG. 9A depicts CSC vascular buds positive for the UEA-I marker and PECAM. FIG. 9B depicts a red channel image of the cells of FIG. 9A showing cells positive for UEA-I. FIG. 9C green channel image of the cells of FIG. 9A showing s cells positive for PECAM. FIG. 9D depicts a blue channel image of the cells of FIG. 8A representing the nuclear stain bisbenzimide.

### DETAILED DESCRIPTION OF THE DISCLOSURE

The present disclosure provides a cell population obtained from human tumors that consist mainly of high purity cancer stem cells and subpopulations thereof responsible for vascular mimicry. Vascular mimicry (VM) cancer stem cells (CSC) are responsible for the vasculogenic-like networks formed by the tumors themselves. In embodiments, the purity of the cell population is at least 50%, at least 60%, at least 70%, at least 80%, at least 90%, at least 95%, or at least 99% cancer stem cells. These cancer stem cells are cancer progenitors and have the capacity of continuous self-renewal and differentiation to a certain level. The disclosure also concerns a method to produce a purified population of VM-CSC, for further use as an antigen source for autologous immune therapy of cancer.

Testing and screening embodiments are also encompassed. The present disclosure uses the high purity VM-CSC population for genetic analysis to identify unique changes that drive the formulation of personalized medicines. The present disclosure provides a novel cell line that is modified in vitro, where this modification enhances the immune stimulatory characteristics of the VM-CSC. The VM-CSC cell line is an improvement over similar technologies using crude tumor preparations, as it provides a superior antigenic signal to noise ratio. The cell line lacks contaminant cell populations, such as fibroblasts, that could alter or diminish the in vitro or in vivo applications. The exemplary cell line of the present disclosure is also used for manufacturing of compositions for treating cancer.

As used herein, the term “derived from,” in the context of peptides derived from one or more cancer cells, encompasses any method of obtaining the peptides from a cancer cell or a population of cancer cells. The cancer cell can be broken, for example, by a homogenizer or by osmotic bursting, resulting in a crude extract. Peptides, oligopeptides, and polypeptides of the crude extract can be exposed to dendritic cells, followed by processing of the peptides by the dendritic cells. The term “derived from” also encompasses intact cancer cells, where the cancer cells are living, or where the cancer cells have been treated with irradiation but are still metabolically active, or where the cancer cells have been treated with a cross-linking agent and therefore still comprise the peptides. “Derived from” also includes mixtures of cancer cell debris, free cancer cell proteins, and irradiated cancer cells, that therefore are derived from the cancer cells. “Derived from” also includes an extract from cancer cells containing messenger RNA (mRNA).

“Administration” as it applies to a human, mammal, mammalian subject, animal, veterinary subject, placebo subject, research subject, experimental subject, cell, tissue, organ, or biological fluid, refers without limitation to contact of an exogenous ligand, reagent, placebo, small molecule, pharmaceutical agent, therapeutic agent, diagnostic agent, or composition to the subject, cell, tissue, organ, or biological fluid, and the like. “Administration” can refer, e.g., to therapeutic, pharmacokinetic, diagnostic, research, placebo, and experimental methods. Administration can refer to in vivo treatment of a human or animal subject. Treatment of a cell encompasses contact of a reagent with the cell, as well as contact of a reagent with a fluid, where the fluid is in contact with the cell. “Administration” also encompasses in vitro and ex vivo treatments, e.g., of a cell, by a reagent, diagnostic, binding composition, or by another cell.

“Effective amount” encompasses, without limitation, an amount that can ameliorate, reverse, mitigate, prevent, or diagnose at least one symptom or sign of a medical condition or disorder. Unless dictated otherwise, explicitly or by context, an “effective amount” is not limited to a minimal amount sufficient to achieve a desired outcome nor limited to the optimal amount sufficient to achieve the desired outcome.

The severity of a disease or disorder, as well as the ability of a treatment to prevent, treat, or mitigate, the disease or disorder (achieve the desired outcome) can be measured, without implying any limitation, by a biomarker or by a clinical parameter. Biomarkers include blood counts, metabolite levels in serum, urine, or cerebrospinal fluid, tumor cell counts, cancer stem cell counts, tumor levels. Tumor levels can be determined by the Response Evaluation Criteria In Solid Tumors (RECIST) criteria (Eisenhauer, et al. (2009) Eur. J. Cancer. 45:228-247). Expression markers encompass genetic expression of mRNA or gene amplification, expression of an antigen, and expression of a polypeptide. Clinical parameters include progression-free survival (PFS), 6-month PFS, disease-free survival (DFS), time to progression (TTP), time to distant metastasis (TDM), and overall survival, without implying any limitation.

A composition that is “labeled” is detectable, either directly or indirectly, by spectroscopic, photochemical, biochemical, immunochemical, isotopic, or chemical methods. For example, useful labels include ³²P, ³³P, ³⁵S, ¹⁴C, ³H, ¹²⁵I, stable isotopes, epitope tags fluorescent dyes, electron-dense reagents, substrates, or enzymes, e.g., as used in enzyme-linked immunoassays, or fluorettes (disclosed in U.S. Pat. No. 6,747,135 which is incorporated by reference herein for all it discloses regarding fluorettes).

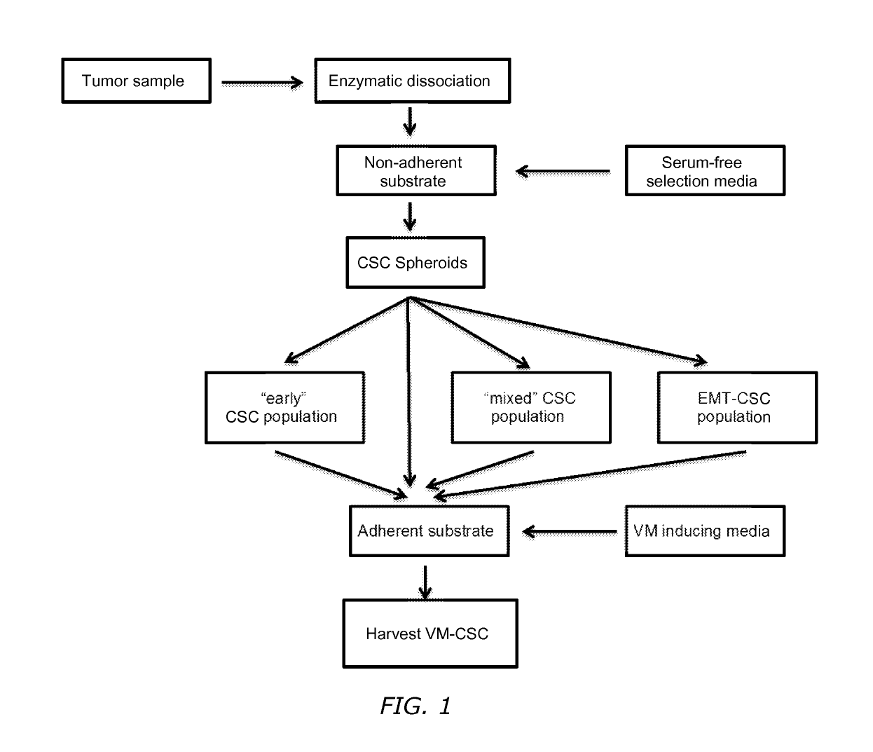

Therefore, disclosed herein are methods for preparing a population of purified spheroids, or single cells preparations derived from spheroids, of cancer stem cells, the method comprising acquiring a biopsy of a tumor, dissociating the cells of the biopsy, in vitro culturing the dissociated cells in a defined medium on a substrate, wherein the defined medium is supplemented with at least one growth factor that acts through the mitogen activated protein kinase (MAPK) pathway to yield a population of purified spheroids, or single cell preparations of cancer stem cells. The defined medium is optionally supplemented with one or more ligands for a receptor tyrosine kinase (RTK). At least about 50%, at least about 60%, at least about 70%, or at least about 80% of the CSC in the population express one or more of the biomarkers ATP-binding cassette sub-family G member 2 (ABCG2; GenBank Accession Number AAG52982.1), CD133, CD24, CD44, CD34, CD117, cytokeratin 8 (CK8), epithelial cell adhesion molecule (EpCAM; GenBank Accession Number NP_{—}002345.2), Ki-67, Nanog (GenBank Accession Number NM_{—}024865.2, NP_{—}079141.20), N-cadherin, neural cell adhesion molecule (NCAM; CD56), Oct3/4 (GenBank Accession Number NP_{—}002692.2; NP_{—}976034.4; NP_{—}001167002.1; NP_{—}068812.10), Slug (SNAI2)/Snail (SNAI1) (Slug/Snail), Twist, vimentin, aldehyde dehydrogenase (ALDH), transforming growth factor beta receptor (TGFβR), Sox2, epidermal growth factor receptor (EGFR), nestin, tumor protein p53 (TP53), vascular endothelial growth factor receptor 1 (VEGF-R1), VEGF-R2, VE-cadherin, vascular endothelial growth factor A (VEGF-A), von Willebrand Factor (vWF), platelet endothelial cell adhesion molecule (PECAM), and Ulex europaeus agglutinin I (UEA-1). A flow chart of the formation of the disclosed cell populations is presented in FIG. 1.

As used herein, the term “spheroids” refers to spherical aggregates of cancer stem cells formed by culture of cancer cells in serum-free medium. The ability to form spheroids is a characteristic of cancer stem cells.

In certain embodiments, at least about 50%, at least about 60%, at least about 70%, or at least about 80% of the cells in the CSC spheroid population express two or more of the biomarkers EpCAM, CD117, ALDH, CD133, CD24, Ki-67, NCAM, vimentin, CK8, TGFβR, EGFR, CD44, ABCG2, Slug/Snail, nestin, and TP53. In other embodiments, at least about 80% of the cells in the CSC spheroid population express two or more of the biomarkers EpCAM, CD117, ALDH, CD133, CD24, and Ki-67. In another embodiment, at least about 90% of the cells in the CSC spheroid population express two or more of the biomarkers EpCAM, CD117, ALDH, CD133, CD24, and Ki-67.

The spheroid population can be further expanded into one of four different subpopulations by altering culture conditions such as media composition and substrate. The characteristics of the bulk tumor, spheroid, early, mixed, EMT-CSC, and VM-CSC populations are presented in Table 1.

TABLE 1
Summary of the conditions used to produce vascular
mimicking (VM) cell populations from bulk tumors

| | | | Markers | Conditions | Usefulness |
|---|---|---|---|---|---|
| Population | Availability | Cell type | (partial list) | for isolation | in therapy |
| Excised | immediate | tumor cells, | Mixed markers from | Lysate and/or | Diluted |
| tumor, bulk | | normal cells, | any of the cell | enzyme- | antigenicity |
| | | few CSC or | populations described | dissociated | |
| | | VM* cells | below | | |
| Spheroids | 7-14 days | CSC** | At least two of | Non-adherent | Proper |
| | | | EpCAM, CD117, | culture | antigenic |
| | | | ALDH, CD133, CD24, | RTK ligands | signal |
| | | | Ki-67 | Serum free | |
| | | | Optionally NCAM, | media | |
| | | | vimentin, CK8, | | |
| | | | TGFβR, EGFR, , | | |
| | | | CD44, ABCG2, | | |
| | | | Slug/Snail, nestin, | | |
| | | | TP53 | | |
| Colonies | 14 days or | CSC (very | At least two of | Adherent | Proper |
| with small | longer | early, | EpCAM, CD133, | culture | antigenic |
| cuboid cells, | | embryonic- | CD44, Nanog, Sox 2, | Serum free | signal |
| “early” | | like) | Oct3/4, CD117, Ki-67 | media, | |
| population | | | Optionally | bFGF | |
| | | | TGFβR, CD24 | Activin A | |
| Epithelial | 14 days or | More or less | Any combination of | Adherent | Proper |
| monolayer, | longer | differentiated | cancer specific | culture | antigenic |
| cells with | | CSC (mixed) | markers or CSC | Optional, | signal |
| various | | | markers | RTK ligands | |
| sizes, small | | | | | |
| cuboid to | | | | | |
| giant cells | | | | | |
| Monolayer | 14 days or | EMT-CSC | At least two of | Adherent | Proper |
| with spindle | and longer | (mesenchyma | NCAM, Slug/Snail, | culture, | antigenic |
| or irregular | | Hike) | Twist, CD24 | bFGF, | signal |
| shaped cells | | | Optionally N-cadherin, | FBS | |
| “EMT” | | | CD44, vimentin, , | | |
| population*** | | | CD133, Nanog, | | |
| | | | CD117 | | |
| Buds, or | 1-3 days | VM-CSC | At least two of | Adherent | Proper |
| strings of | | | VEGF-R2, VE- | culture or | antigenic |
| VM cells | | | Cadherin, VEGF-A, | suspension | signal |
| | | | CD34, VWF, PECAM | Soluble | |
| | | | Optionally VEGF-R1, | laminin with | |
| | | | UEA-I | alpha chain 1, | |
| | | | | 2, 3 or 4 | |

*VM = vascular mimicking cells;
**CSC = cancer stem cell;
***EMT = epithelial to mesenchymal transition

Furthermore, any of the early CSC, mixed CSC, EMT-CSC, or VM-CSC populations can be obtained from CSC spheroids, early CSC, mixed CSC, or EMT-CSC by changing the media and conditions as disclosed in Table 1.

In one embodiment, the CSC spheroids are further cultured on an adherent substrate in the presence of activin A, FGF, and a serum-free media (selection media) to yield colonies with small cells referred to herein as an “early” population of CSC which have characteristics of embryonic stem cells, and at least about 50%, at least about 60%, at least about 70%, or at least about 80% of the cells in the early CSC population express two or more of biomarkers EpCAM, CD133, CD44, Nanog, Sox2, Oct3/4, CD117, and Ki-67. In another embodiment, at least about 80% of the cells in the early CSC population express two or more of biomarkers EpCAM, CD133, CD44, Nanog, Sox2, Oct3/4, CD117, Ki-67, TGFβR, and CD24. In another embodiment, at least about 90% of the cells in the early CSC population express two or more of biomarkers EpCAM, CD133, CD44, Nanog, Sox2, Oct3/4, CD117, and Ki-67.

In another embodiment, the CSC spheroids are further cultured on an adherent substrate under low calcium conditions in a serum-containing (expansion media) to yield colonies mixed with a monolayer wherein the cells have heterogeneous morphologies. The culture medium optionally includes one or more RTK ligands. These cells are referred to herein as a “mixed” population of CSC which have a mixed differentiation profile, and at least about 50%, at least about 60%, at least about 70%, or at least about 80% of the cells in the mixed CSC population express two or more of biomarkers ABCG2, CD133, CD24, CD44, CD34, CD117, CK8, EpCAM, Ki-67, Nanog, N-cadherin, NCAM, Oct3/4, Slug/Snail, Twist, vimentin, ALDH, TGFβR, Sox2, EGFR) nestin, TP53, VEGF-R1, VEGF-R2, VE-cadherin, VEGF-A, vWF, PECAM, and UEA-1. In another embodiment, at least about 90% of the cells in the mixed CSC population express two or more of biomarkers ABCG2, CD133, CD24, CD44, CD34, CD117, CK8, EpCAM, Ki-67, Nanog, N-cadherin, NCAM, Oct3/4, Slug/Snail, Twist, vimentin, ALDH, TGFβR, Sox2, EGFR) nestin, TP53, VEGF-R1, VEGF-R2, VE-cadherin, VEGF-A, vWF, PECAM, and UEA-1.

In yet another embodiment, the CSC spheroids are further cultured on an adherent substrate in the presence of FGF and a serum-containing media (expansion media) to yield a monolayer of spindle- or irregularly-shaped cells referred to herein as mesenchymal-like CSC or “EMT-CSC” (epithelial to mesenchymal transitioned [EMT] cancer stem cells). In this population, the spheroids have undergone a process of EMT characterized by the loss of the expression of at least one of the epithelial biomarkers Ki-67 and EpCAM. As used herein, loss of the expression of a biomarker refers to undetectable expression or expression in 40% (or less) of the cells, expression in 30% (or less) of the cells, expression in 20% (or less) of the cells, or expression in 10% (or less) of the cells. Additionally, the EMT process is characterized by the increase in the expression of at least one of the mesenchymal biomarkers Slug/Snail, Twist, CD44, NCAM, N-cadherin, and vimentin to at least 30%, at least 40%, at least 50%, at least 60%, at least 70%, at least 80%, at least 90%, at least 95%, or at least 99% of the cells in the population expressing the biomarker(s) of interest.

In one embodiment, at least about 50%, at least about 60%, at least about 70%, or at least about 80% of the cells in the EMT-CSC population express two or more of the biomarkers NCAM, Slug/Snail, CD24, and Twist. In yet another embodiment, at least about 80% of the cells in the EMT-CSC population express two or more of the biomarkers NCAM, Slug/Snail, CD24, Twist, N-cadherin, CD44, vimentin, CD133, Nanog, and CD117. In yet another embodiment, at least about 90% of the cells in the EMT-CSC population express two or more of the biomarkers NCAM, Slug/Snail, CD24, and Twist.

In another embodiment, the CSC spheroids, early CSC, mixed CSC, or EMT-CSC are further cultured on an adherent substrate or in suspension in the presence of soluble laminin (any soluble laminin including one of the α1, α2, α3 or α4 chains) and a serum-containing media (expansion media) to yield buds or strings of cells (VM cells). These cells exhibit the vascular mimicry profile, and at least about 50%, at least about 60%, at least about 70%, or at least about 80% of the cells in the VM-CSC population express two or more of biomarkers VEFG-R2, VE-cadherin, VEGF-A, CD34, vWF, PECAM, VEGF-R1, and UEA-1. In another embodiment, at least about 80% of the cells in the VM-CSC population express two or more of biomarkers VEFG-R2, VE-cadherin, VEGF-A, CD34, vWF, and PECAM. In another embodiment, at least about 90% of the cells in the VM-CSC population express two or more of biomarkers VEFG-R2, VE-cadherin, VEGF-A, CD34, vWF, and PECAM.

In certain embodiments of the cell populations, the cells express one or more of the indicated biomarkers. In other embodiments, the cells express two or more, three or more, four or more, five or more, six or more, seven or more, eight or more, nine or more, or ten or more of the indicated biomarkers. In yet other embodiments, the cells express 1, 2, 3, 4, 5, 6, 7, 8, 9, 10, 11, 12, 13, 14, 15, 16, 17, 18, 19, 20, or more of the indicated biomarkers.

Expression of biomarkers by a single cell, by a population of cells, or by a population of cells located in a specific structure such as a monolayer or a spheroid, can be determined by measuring expression of the polypeptide form of the biomarker or the mRNA form of the biomarker. Polypeptide expression can be measured using a labeled antibody, while nucleic acid expression can be measured by hybridization techniques, are available to the skilled artisan. Biomarkers that are not polypeptides or nucleic acids, such as oligosaccharides or small molecule metabolites, can also be measured by methods available to the skilled artisan.

Also disclosed herein are methods to obtain pure populations of isolated CSC from tumor samples of various sizes (1 mg to grams). The tumor samples can be fresh or frozen, are dissociated by mechanical and/or enzymatic treatment, or are cultivated directly with minimal mechanical fragmentation.

The tumor cells can be from any tumor which exhibits vascularization including, but not limited to, adrenocortical carcinoma, anal cancer, appendix cancer, astrocytoma, basal-cell carcinoma, bile duct cancer, bladder cancer, bone cancer, brain cancer, breast cancer, bronchial adenomas/carcinoids, carcinoid tumor, cervical cancer, chronic myeloproliferative disorders, colon cancer, desmoplastic small round cell tumor, endometrial cancer, ependymoma, esophageal cancer, Ewing's sarcoma, germ cell tumors, eye cancer, gallbladder cancer, gastric cancer, gastrointestinal carcinoid tumor, gastrointestinal stromal tumor (GIST), gestational trophoblastic tumor, glioma, gastric carcinoid, head and neck cancer, heart cancer, hepatocellular cancer, Hodgkin lymphoma, hypopharyngeal cancer, islet cell carcinoma, Kaposi sarcoma, kidney cancer, leukemias, lip and oral cavity cancer, liposarcoma, liver cancer, lung cancer, lymphomas, macroglobulinemia, medulloblastoma, melanoma, merkel cell carcinoma, mesothelioma, mouth cancer, multiple myeloma/plasma cell neoplasm, mycosis fungoides, nasal cavity and paranasal sinus cancer, nasopharyngeal carcinoma, neuroblastoma, oral cancer, oropharyngeal cancer, ovarian cancer, ovarian epithelial cancer, pancreatic cancer, parathyroid cancer, penile cancer, pharyngeal cancer, pheochromocytoma, pineal astrocytoma, pineal germinoma, pineoblastoma, pituitary adenoma, pleuropulmonary blastoma, prostate cancer, rectal cancer, renal cell carcinoma, retinoblastoma, rhabdomyosarcoma, salivary gland cancer, sarcoma, Sézary syndrome, skin cancer, squamous cell carcinoma, stomach cancer, testicular cancer, throat cancer, thymoma, thyroid cancer, urethral cancer, uterine cancer, uterine sarcoma, vaginal cancer, vulvar cancer, Waldenström macroglobulinemia, or Wilms tumor.

Also disclosed herein, a non-adherent substrate is any biocompatible material with anti-biofouling properties or a coating with anti-biofouling properties (reduces accumulation of cells on a wetted surface) applied to a common culture surface. The coating can be applied using coating agents such as amino-silanes. If there is a non-adherent or anti-biofouling substrate, this substrate can be used for about 0-25 days, such as 0-21 days, 5-20 days, 5-10 days, 10-20 days, or any time period between zero and 25 days.

In another embodiment of the method that uses an adherent substrate, the adherent substrate can be one that is rich in RGD (Arg-Gly-Asp) tripeptide motifs (e.g., collagen, gelatin, MATRIGEL®). An adherent substrate is a surface that is configured to adhere to, and to collect, anchorage dependent cells. Moreover, the substrate can be an adherent substrate that is configured to adhere to and to collect anchorage dependent cells that are fibroblasts. RGD peptides can also be grafted on polymeric backbones such as polystyrene, hyaluronan, poly-lactic acid, or combinations thereof. The backbone can further carry proteoglycans. The proteoglycans can carry growth factors such as fibroblast growth factor (FGF), epidermal growth factor (EGF), activin A or follistatin.

Additionally, for generating VM-CSC, the culture surface can optionally be coated with fibronectin or poly-L-ornithine.

A non-adherent substrate can cause fast and efficient enrichment of the cultures with cancer stem cells. In certain culture embodiments, a first period of culture is provided on an adherent substrate, followed by a second period of culture on a non-adherent substrate. Also provided is a first period of culture on a non-adherent substrate, followed by a second period of culture on an adherent substrate. Periods can be, for example, one half day, 1 day, 2 days, 3 days, 4 days, 5 days, 6 days, 7 days, 8 days, 9 days, 10 days, 11 days, 12 days, 13 days, 14 days, 15 days, and the like, or any range thereof, such as 2-4 days, or 8-10 days, and so on. Additionally, the cycle can repeat such as an adherent culture followed by a non-adherent culture followed by an adherent culture, etc. In another embodiment, the cycle can repeat such as a non-adherent culture followed by an adherent culture, followed by a non-adherent culture, etc.

In another embodiment, the defined medium is supplemented with at least one growth factor that acts through the mitogen activated protein kinase (MAPK) pathway. In one embodiment, the growth factor is one or both of FGF and EGF, or an analogue thereof. In one embodiment, the FGF is basic fibroblast growth factor (bFGF). In another embodiment, the defined medium is supplemented with activin A. In another embodiment, the defined medium is not supplemented with activin A. Also disclosed is a defined medium supplemented with an agonist of activin A, in amount effective to prevent spontaneous differentiation of CSC. In another embodiment, the defined medium is supplemented with at least one ligand for the receptor tyrosine kinases (RTK). Ligands for RTKs are soluble or membrane-bound peptide/protein hormones including nerve growth factor (NGF), platelet derived growth factor (PDGF), FGF, EGF, and insulin.

Also provided is a VM-CSC cell line that is unique to each patient obtained from the patient's primary tumor.

The present disclosure encompasses nucleic acids, gene products, polypeptides, and peptide fragments, where identity can be reasonably established by a trivial name alone. Also encompassed, are nucleic acids, gene products, polypeptides, and peptide fragments, based on a particular GenBank Accession No., where the nucleic acid, polypeptide, and the like, has at least 50% sequence identity, at least 60%, at least 70%, at least 80%, at least 90%, at least 95%, at least 98%, at least 99%, or 100% identity sequence identity, to that of the GenBank No. where the biochemical function, or physiological function are shared, at least in part, or alternatively, irrespective of function.

Provided is a method wherein an immune response to cancer in a subject is stimulated with one of the compositions disclosed herein. The immune response that is stimulated comprises one or more of CD4⁺ T cell response, CD8⁺ T cell response, and B cell response. In certain embodiments, the CD4⁺ T cell response, CD⁺ T cell response, or B cell response, can be measured by ELISPOT assays, by intracellular cytokine staining (ICS) assays, by tetramer assays, or by detecting antigen-specific antibody production, according to assays that are known by persons of ordinary skill in the art. The immune response can comprise a survival time such as a 2-year overall survival (OS), and where the 2-year overall survival is at least 60%. An immune response in a patient can also be assessed by endpoints that are used in oncology clinical trials, including objective response (RECIST criteria), overall survival, progression-free survival (PFS), disease-free survival, time to distant metastasis, 6-month PFS, 12-month PFS, and so on.

Also disclosed herein are dendritic cells stimulated ex vivo with the VM-CSC, or antigens derived therefrom, for use in therapy of cancer. Encompassed herein are immunogenic compositions, such as vaccine compositions, comprising dendritic cells loaded with (exposed to) the VM-CSC ex vivo. In certain embodiments, the dendritic cells and tumor cells are from the same human subject (autologous), although embodiments where the dendritic cells and VM-CSC are from different, HLA-matched subjects are within the scope of the present disclosure. In other embodiments, the donors of the tumor cells and the dendritic cells are HLA-matched. In yet another embodiment, the donor of the immunogenic composition and the recipient thereof are HLA-matched.

Dendritic cells can be loaded with tumor cell antigens comprising whole cells, cell lysates, cell extracts, irradiated cells or any protein derivative of a tumor cell, such as a VM-CSC. Dendritic cell immunogenic compositions can be prepared, and administered to a human subject by one or more routes of administration as are known to persons of ordinary skill in the art.

In certain embodiments, the VM-CSC cells are irradiated, or otherwise treated to prevent cell division, prior to loading with the dendritic cells. Alternatives to radiation include nucleic acid cross-linking agents that prevent cell division. Also provided is a method that uses of the VM-CSC population, as disclosed above, as a source of antigen for autologous immune therapy, for example, where the CSC are inactivated by a radiant energy (e.g., gamma, UV, X), temperature (e.g., heat or cold), or chemical (e.g., cytostatic, aldehyde, alcohol) methods, or combinations thereof. In other embodiments, the VM-CSC are used as a source of antigen for ex vivo activation of dendritic cells.

The present disclosure provides prepared VM cells (VM-CSC), provides DC loaded with the prepared VM-CSC, and provides immunogenic compositions (or vaccines) comprising dendritic cells loaded the prepared VM-CSC. Without implying any limitation, an immunogenic composition of the present disclosure can comprise DC loaded with VM-CSC spheroids, loaded with a population of cells that comprises spheroids, loaded with a population of cells that was derived from spheroids and that were expanded on an adherent surface prior to loading on DC, loaded with spheroids that were subjected to homogenization or sonication prior to loading on DC, loaded with a population of expanded cells that were subjected to homogenization or sonication prior to loading on DC, and so on.

Also disclosed herein is a population of VM-CSC that is capable of stimulating an effective immune response against a cell expressing at least one tumor-specific antigen, wherein the VM-CSC population is contacted with at least one dendritic cell, wherein the VM-CSC population is processed in vivo or ex vivo by the dendritic cell, and wherein an effective immune response occurs in the subject in response to administration of the at least one dendritic cell to a subject.

An immune stimulatory amount of the disclosed compositions is, without limitation, an amount that increases ELISPOT assay results by a measurable amount, that increases ICS assay results by a measurable amount, that increases tetramer assay results by a measurable amount, that increases the blood population of antigen-specific CD4+ T cells by a measurable amount, that increases the blood population of antigen-specific CD8+ T cells by a measurable amount, or where the increase is by at least 10%, 20%, 30%, 40%, 50%, 60%, 70%, 80%, 90%, 100%, 1.5-fold, 2.0-fold, 3.0-fold, and the like, when compared to a suitable control. A suitable control can be a control composition, where dendritic cells are not loaded with VM-CSC cells, or are not loaded with peptide derived from VM-CSC cells.

The disclosure also provides pharmaceuticals, reagents, kits including diagnostic kits, that wherein the pharmaceuticals, reagents, and kits, comprise dendritic cells (DC), antibodies, or antigens. Also provided are methods for administering compositions that comprise at least one dendritic cell and at least one antigen, methods for stimulating antibody formation, methods for stimulating antibody-dependent cytotoxicity (ADCC), methods for stimulating complement-dependent cytotoxicity, and methods and kits for determining patient suitability, for determining patient inclusion/exclusion criteria in the context of a clinical trial or ordinary medical treatment, and for predicting response to the pharmaceutical or reagent. The pharmaceutical compositions, reagents, and related methods, of the disclosure encompass CD83 positive dendritic cells, where CD83 is induced by loading with IFN-gamma-treated, or untreated, cancer cells. In a CD83 aspect of the disclosure, the CD83 is induced by at least 2%, at least 3%, at least 4%, 6%, 7%, 8%, 9%, 10%, and the like. In another aspect, excluded are DC reagents, or DC-related methods, where CD83 on dendritic cells is not detectably induced by loading with IFN-gamma.

In one embodiment, a kit is provided which includes all of the reagents for generating CSC spheroids, early CSC, mixed CSC, EMT-CSC, and/or VM-CSC from tumor samples according to the methods disclosed herein and/or reagents for characterizing the CSC spheroids, early CSC, mixed CSC, EMT-CSC, and/or VM-CSC, and instructions for generating and/or characterizing CSC spheroids, early CSC, mixed CSC, EMT-CSC, and/or VM-CSC. In another embodiment, the kit additionally, or alternatively, includes reagents and instructions for isolating dendritic cells, for loading the dendritic cells with VM-CSC, and/or for administering the DC-VM-CSC composition to a subject.

Additionally, cancer stem cells responsible for VM can be used for in vitro assays for anti-angiogenic drug therapies or as diagnostic tool for determining the severity of patient's cancer. Furthermore, what is presently disclosed is the use of such cell line for manufacturing of a drug used for treatment of a wide variety of cancers due to overlapping mechanisms underlying VM in different cancers.

Tumor Sample Processing

The cancer stem cell population of the present disclosure can originate from fresh or frozen samples of patient tumor. The tumor sample can be a biopsy, a lavage of a body cavity containing tumor cells, a fine needle aspiration, ascites fluid, or any other tissue sample that contains tumor cells.

The tumor sample may be transported in a generic buffered media with a pH of about 7.4 (+/−0.6) such as RPMI, DMEM, F12, Williams, or combinations containing a protein source such as animal or human serum in concentrations from 0 to 100% or albumin at concentrations from 0 to 0.5% or macromolecules that ensure a physiological osmotic pressure. Examples of natural or artificial macromolecules are, but not limited to, hyaluronan, dextrans, and polyvinyl alcohol. An antibiotic such as penicillin, streptomycin, gentramicyn in an optional combination with an antifungal such as amphotericin B, FUNGIZONE® (Life Technologies, Carlsbad, Calif.), can be used in the media to provide antimicrobial properties and reduce the risk of contamination during transportation.

The tumor sample can be kept below a metabolic active state by reducing the media temperature to 2 to 30° C., thus allowing the viability maintenance for a limited time (between 0 to 72 hours) before processing. Packaging (e.g., insulated packaging) may be used to ensure the proper temperature control during transportation.

The solid tumor tissue is then processed by mechanical dissociation using a sharp blade or tissue grinder device into small, less than 1 mm (on any dimension) fragments.

The solid tissue is optionally further processed by enzymatic dissociation. A variety of enzymes can be used to isolate single cells. Nonspecific proteolytic enzymes such as trypsin and pepsin can be used successfully. Targeting minimal cell membrane damage specific enzymes, including collagenase, dispase, elastase, hyaluronidase, or combinations thereof, may be used in the disclosed methods. Deoxyribonuclease (DNAse) can be used to degrade the free DNA from cell detritus responsible for unwanted stickiness of the cell preparation. After dissociation, the cells in suspension are washed from the excess enzyme and debris by straining through a 50-100 μm mesh and repeated centrifugation in a buffered saline (PBS, HBSS) or cell culture media.

Cell Culture Conditions and Spheroid Production

The single cell suspension described above is transferred in culture conditions that promote isolation, expansion of the stem cells and suppression of the differentiated and/or normal cells. This is accomplished by the congruence of the physical conditions, chemical environment, and manipulations.

The cell suspension is exposed to a non-adherent (anti-biofouling) substrate that does not allow cell attachment. Mature cells are commonly anchorage dependent and are rapidly eliminated when a proper adherent substrate is not provided. An anti-biofouling substrate can employ commercial products such as ultralow adherent flasks (Corning, Corning, N.Y.), polymers with natural hydrophobic properties (polyvinyl, polyethylene, polypropylene, fluoro-polymers) or coating with natural carbohydrate polymers such as agar-agar, starch, and the like.

The cancer stem cells will aggregate and/or clonally expand in spheroid formations that contain high purity cancer stem cells. The mature cells will remain isolated and non-adherent. A differential gravitational separation can be used to select the larger spheroids from single cells, by simply allowing a timed vertical sedimentation or a short time low force centrifugation (less than 100×G). The selection method described is designed to accomplish the following: (a) eliminate of anchorage dependent cells that are, in general, mature, normal cells; (b) promote the clonal expansion in small clumps or spheroids of the young, stem cells that are anchorage independent; (c) promote the local autocrine activity as a result of clonal expansion of the stem cells; and (d) eliminate the autocrine source of activin A that is secreted by normal fibroblasts or cancer cells.

The ability of cells to form spheres results, in part, from cell-surface proteins called integrins. Homophilic integrins expressed on the cell's surface ensure that cells of the same type “stay together”. Spheres are formed directly from enzyme digest which is a single cell suspension at the very beginning of a culture, or can be formed from frozen sample or an existing attached culture at any time. The enzyme digest seeding result in this spherical formations that incorporate the cells with the specific surface properties.

Fibroblasts, for example, are not incorporated into spheroids and are removed from a culture during gravitational feeding. The media used lacks molecules that promote adhesion in order to prevent the non-specific agglomeration of the cells not having homophilic proprieties and to prevent the adhesion to the culture vessel surfaces. Such cell adhesion molecules (CAMs) are commonly found in the animal or human serum. Therefore a media composition which is serum-free is suitable for culture of non-adherent spheroids.

In the serum-free media culture, supplements to the media may include any hormones, nutrients, mineral, and vitamins that are required for supporting growth and maintenance, or other desired aspects of cell physiology and function. In some instance one can stimulate and sustain the stem cell proliferation with the addition or adjustment of amount of growth factors that possess a mitogenic activity, such as the FGF family and EGF.

Spheres of cells (spheroids), including spheres of cancer stem cells, can be characterized in terms of biomarker expression by way of fixing and staining with labeled antibodies, followed by viewing with confocal microscopy. Biomarkers may also be measured by other immunochemistry methods, e.g., flow cytometry. Spheres can be prepared, for example, from suspensions obtained from fresh tumors, or from cells adapted to grow as adherent cells. The morphology of spheres, for example, large and irregular versus tiny and compact, may be influenced by the choice of medium.

In another embodiment, a cell population adherent to the anti-biofouling coating can be isolated based on aberrant activation of sonic hedgehog signaling mediated by protein kinase B (AKT) and focal adhesion kinase (FAK) signaling. These phenomena can be enhanced by modifications of the membranes induced by enzymes such as metalloproteases or enzymes used in dissociation (trypsin/collagenase). Such cell population can be associated with rapid proliferative and invasive tumors. Methods for assessing normal or aberrant activation of the sonic hedgehog signaling are available and known to persons of ordinary skill in the art.

Medium Used in Cell Culture

The defined media that is used to isolate the CSC promotes cell survival and is specifically formulated for selection. The media is rich in carbohydrates and lipids but has minimal amount of protein (0.1%-3% albumin or 1%-5% serum). It contains not more than 1.5 mMol total calcium, does not contain inorganic iron compounds; rather, iron is completely bound to a transporter such as transferrin. The media is provided with an excess of essential and non-essential amino acids and essential lipids (alpha-linolenic and linoleic acids) (Table 4). Optionally, the media does not contain activin A and may contain an activin A receptor blocker such as follistatin. Also optionally, the media does not contain antioxidants such as superoxide dismutase (SOD) or catalase, but contains thiolic antioxidants such as glutathione.

The culture media consists in a basal formulation such as DMEM, F12, Williams, RPMI, Lebovitz supplemented with proteins (in certain formulations), amino acids, antioxidants, energetic substrate (glucose, galactose, L-glutamine), vitamins (B12), hormones (thyroid hormones, insulin) and growth factors (FGF, EGF) as depicted in Table 2.

The protein can be albumin in concentration of 0.1-0.5%, fetal bovine serum (FBS) 0.5%-20%. The protein can be substituted with macromolecules such as dextrans, hyaluronan, poly-vinyl alcohol in concentration ranging from 0.1% to 0.5%. The composition of such media is listed in Table 2, Table 3, and Table 4. The supplements are added into the media and mixed for feeding the cell cultures.

The media can be replaced in a three day a week schedule (e.g., Monday-Wednesday-Friday), or more frequently, e.g., every other day or daily, if the expansion is fast. A continuous feed or a micro-batch feed bioreactor can be used in the expansion phase.

The media contains growth factors that act through the MAPK pathway such as FGF and EGF. The concentration of these growth factors can vary between 0.1 to 100 ng/mL, commonly around 10 ng/mL.

In one embodiment, the media is supplemented with FGF at about 0.1 to 100 ng/mL, at about 0.5-50 ng/mL, at about 1-40 ng/mL, at about 2-30 ng/mL, at about 3-20 ng/mL, at about 5-15 ng/mL, at about 6-14 ng/mL, at about 7-13 ng/mL, at about 8-12 ng/mL, at about 9-11 ng/mL, or at about 10 ng/mL. In other embodiments FGF is present in the media at about 5 ng/mL, at about 6 ng/mL, at about 7 ng/mL, at about 8 ng/mL, at about 9 ng/mL, at about 11 ng/mL, at about 12 ng/mL, at about 12 ng/mL, at about 14 ng/mL, or at about 15 ng/mL.

In another embodiment, the media is supplemented with EGF at about 0.1 to 100 ng/mL, at about 0.5-50 ng/mL, at about 1-40 ng/mL, at about 2-30 ng/mL, at about 3-20 ng/mL, at about 5-15 ng/mL, at about 6-14 ng/mL, at about 7-13 ng/mL, at about 8-12 ng/mL, at about 9-11 ng/mL, or at about 10 ng/mL. In other embodiments EGF is present in the media at about 5 ng/mL, at about 6 ng/mL, at about 7 ng/mL, at about 8 ng/mL, at about 9 ng/mL, at about 11 ng/mL, at about 12 ng/mL, at about 12 ng/mL, at about 14 ng/mL, or at about 15 ng/mL.

Also provided is a medium which is not supplemented with one or both of superoxide dismutase (SOD) or catalase. The use of antioxidants can have both positive and negative consequences. Cancer stem cells are far more tolerant than normal cells to free radicals and glycolytic metabolism. Therefore in suboptimal cultures such as high density, infrequent media replacement, high concentration of metabolites in the media, it is most likely that the normal sensitive cells to be eliminated first. By not including antioxidants in the media, a population of cells can be selected that is likely to be of a cancerous origin, more resistant than the normal cells. Therefore, in certain embodiments, antioxidants, such as catalase and inhibitors of SOD are added to the culture medium and in other embodiments, these compounds are omitted from the culture media.

TABLE 2
Basal media composition options for cancer stem cells:

| | M.W. | DMEM/F12 (1:1) | | William's E | | DMEM | | RPMI | | F12 | |
|---|---|---|---|---|---|---|---|---|---|---|---|
| Components | g/mole | mg/L | mM | mg/L | mM | mg/L | mM | mg/L | mM | mg/L | mM |

Amino Acids

| | | | | | | | | | | | |
|---|---|---|---|---|---|---|---|---|---|---|---|
| L-Alanine | 89.10 | 4.45 | 0.05 | 90 | 1.010 | | | | | 8.9 | 0.100 |
| L-Arginine | 174.20 | | | 50 | 0.287 | | | | | | |
| L-Arginine•HCl | 210.65 | 147.5 | 0.70 | | | 84 | 0.399 | 200 | 0.949 | 211 | 1.002 |
| L-Aspara- | 150.10 | 7.50 | 0.05 | 20 | 0.133 | | | 50 | 0.333 | 15.01 | 0.100 |
| gine•H₂O | | | | | | | | | | | |
| L-Aspartic Acid | 133.10 | 6.65 | 0.05 | 30 | 0.225 | | | 20 | 0.150 | 13.3 | 0.100 |
| L-Cysteine | 121.16 | | | 40 | 0.330 | | | | | | |
| L-Cyste- | 175.65 | 17.56 | 0.10 | | | | | | 0.000 | 35.12 | 0.200 |
| ine•HCl•H₂O | | | | | | | | | | | |
| L-Cystine•2HCl | 313.11 | 31.29 | 0.10 | 26.07 | 0.083 | 62.57 | 0.200 | 65.15 | 0.208 | | |
| L-Glutamic Acid | 147.10 | 7.35 | 0.05 | 50 | 0.340 | | | 20 | 0.136 | 14.7 | 0.100 |
| L-Glutamine | 146.10 | 365 | 2.50 | 292 | 1.999 | 584 | 3.997 | 300 | 2.053 | 146 | 0.999 |
| L-Glycine | 75.10 | 18.75 | 0.25 | 50 | 0.666 | 30 | 0.399 | 10 | 0.133 | 7.5 | 0.100 |
| L-Histidine | 155.16 | | | 15 | 0.097 | | | | | | |
| L-Histi- | 209.65 | 31.48 | 0.15 | | | 42 | 0.200 | 15 | 0.072 | 20.96 | 0.100 |
| dine•HCl•H₂O | | | | | | | | | | | |
| L-Hydroxyproline | 131.13 | | | | | | | 20 | 0.153 | | |
| L-Isoleucine | 131.20 | 54.47 | 0.42 | 50 | 0.381 | 105 | 0.800 | 50 | 0.381 | 3.94 | 0.030 |
| L-Leucine | 131.20 | 59.05 | 0.45 | 75 | 0.572 | 105 | 0.800 | 50 | 0.381 | 13.1 | 0.100 |
| L-Lysine•HCl | 182.65 | 91.25 | 0.50 | 87.46 | 0.479 | 146 | 0.799 | 40 | 0.219 | 36.5 | 0.200 |
| L-Methionine | 149.20 | 17.24 | 0.12 | 15 | 0.101 | 30 | 0.201 | 15 | 0.101 | 4.48 | 0.030 |
| L-Phenylalanine | 165.20 | 35.48 | 0.21 | 25 | 0.151 | 66 | 0.400 | 15 | 0.091 | 4.96 | 0.030 |
| L-Proline | 115.10 | 17.25 | 0.15 | 30 | 0.261 | | | 20 | 0.174 | 34.5 | 0.300 |
| L-Serine | 105.10 | 26.25 | 0.25 | 10 | 0.095 | 42 | 0.400 | 30 | 0.285 | 10.5 | 0.100 |
| L-Threonine | 119.10 | 53.45 | 0.45 | 40 | 0.336 | 95 | 0.798 | 20 | 0.168 | 11.9 | 0.100 |
| L-Tryptophan | 204.20 | 9.02 | 0.04 | 10 | 0.049 | 16 | 0.078 | 5 | 0.024 | 2.04 | 0.010 |
| L-Tyro- | 261.20 | 55.79 | 0.21 | 50.65 | 0.194 | 103.8 | 0.397 | 28.83 | 0.110 | 7.8 | 0.030 |
| sine•2Na•2H₂O | | | | | | | | | | | |
| L-Valine | 117.10 | 52.85 | 0.45 | 50 | 0.427 | 94 | 0.803 | 20 | 0.171 | 11.7 | 0.100 |

Sugar

| | | | | | | | | | | | |
|---|---|---|---|---|---|---|---|---|---|---|---|
| D-Glucose | 180.00 | 3151 | 17.51 | 2000 | 11.111 | 4500 | 25 | 2000 | 11.11 | 1802.00 | 10.01 |

Vitamins/Nucleotides/Minute Organics

| | | | | | | | | | | | |
|---|---|---|---|---|---|---|---|---|---|---|---|
| Ascorbic acid | 176.13 | | | 2 | 1.14E−02 | | | | | | |
| Vitamin B-12 | 1355 | 0.6800 | 5.02E−04 | 0.2 | 1.48E−04 | | | 0.005 | 3.69E−06 | 1.4 | |
| (cobalamin) | | | | | | | | | | | |
| Biotin | 244.00 | 0.0035 | 1.43E−05 | 0.5 | 2.05E−03 | | | 0.2 | 8.20E−04 | 0.0073 | 2.99E−05 |
| Choline | 140.00 | 8.98 | 6.41E−02 | 1.5 | 1.07E−02 | 4 | 2.86E−02 | 3 | 2.14E−02 | 13.96 | 0.099714 |
| chloride | | | | | | | | | | | |
| Ergocalciferol | 396.66 | | | 0.1 | 2.52E−04 | | | | | | |
| Folic acid | 441.00 | 2.65 | 6.01E−03 | 1 | 2.27E−03 | 4 | 9.07E−03 | 1 | 2.27E−03 | 1.3 | 2.95E−03 |
| I-inositol | 180.00 | 12.60 | 7.00E−02 | 2 | 1.11E−02 | 7.2 | 4.00E−02 | 35 | 1.94E−01 | 18 | 0.1 |
| Menadione | | | | 0.01 | | | | | | | |
| sodium | | | | | | | | | | | |
| bisulfate | | | | | | | | | | | |
| Niacinamide | 122.00 | 2.02 | 1.66E−02 | 1 | 8.20E−03 | 4 | 3.28E−02 | 1 | 8.20E−03 | 0.037 | 3.03E−04 |
| D-Calcium | 477.00 | 2.21 | 4.63E−03 | 1 | 2.10E−03 | 4 | 8.39E−03 | 0.25 | 5.24E−04 | 0.48 | 1.01E−03 |
| pantothenate | | | | | | | | | | | |
| Pyridoxal HCl | 204.00 | 2.00 | 9.80E−03 | 1 | 4.90E−03 | 4 | 1.96E−02 | | | | |
| Pyridoxine HCl | 206.00 | 0.03 | 1.50E−04 | | | | | 1 | 4.85E−03 | 0.062 | 3.01E−04 |
| Riboflavin | 376.00 | 0.22 | 5.82E−04 | 0.1 | 2.66E−04 | 0.4 | 1.06E−03 | 0.2 | 5.32E−04 | 0.038 | 1.01E−04 |
| Thiamine HCl | 337.00 | 2.17 | 6.44E−03 | 1 | 2.97E−03 | 4 | 1.19E−02 | 1 | 2.97E−03 | 0.34 | 1.01E−03 |
| (Vitamin B1) | | | | | | | | | | | |
| Thymidine | 242.23 | 0.37 | 2.07E−03 | | | | | | | 0.7 | |
| Putres- | 88.15 | 0.08 | 9.19E−04 | | | | | | | 0.161 | |
| cine•2HCl | | | | | | | | | | | |
| Sodium pyruvate | 110.00 | 55.00 | 5.00E−01 | 25 | 0.227 | | | | | 110 | |
| a-Tocopherol | | | | 0.01 | | | | | | | |
| phosphate | | | | | | | | | | | |
| Lipoic acid | 206.00 | 0.11 | 5.10E−04 | | | | | | | 0.21 | |
| Linoleic acid | 280.48 | 0.04 | 1.50E−04 | | | | | | | 0.08 | |
| Para- | | | | | | | | 1 | | | |
| aminobenzoic | | | | | | | | | | | |
| acid | | | | | | | | | | | |
| Vitamin A | | | | 0.1 | | | | | | | |
| acetate | | | | | | | | | | | |

Inorganic Bulk Salts, buffers

| | | | | | | | | | | | |
|---|---|---|---|---|---|---|---|---|---|---|---|
| Magnesium | 95.21 | 28.64 | 0.30 | | | | | | | 57.22 | 0.601 |
| chloride, | | | | | | | | | | | |
| anhydrous | | | | | | | | | | | |
| Magnesium | 120.40 | 48.84 | 0.41 | 97.67 | 0.81 | 97.67 | 0.8112 | 48.84 | 0.41 | | |
| sulfate, | | | | | | | | | | | |
| anhydrous | | | | | | | | | | | |
| Potassium | 74.55 | 311.80 | 4.18 | 400 | 5.37 | 400 | 5.3655 | 400 | 5.37 | 223.6 | 3.00 |
| chloride | | | | | | | | | | | |
| Sodium | 142.00 | 71.02 | 0.50 | | | | | 800 | 5.63 | | |
| phosophate, | | | | | | | | | | | |
| dibasic, | | | | | | | | | | | |
| anhydrous | | | | | | | | | | | |
| Sodium | 160.00 | | | | | 125 | 0.7813 | | | | |
| phosophate | | | | | | | | | | | |
| dibasic•H₂O | | | | | | | | | | | |
| Sodium | 58.44 | 6999.50 | 119.77 | 6800 | 116.36 | 6400 | 109.5140 | 6000 | 102.67 | 7599 | 130.03 |
| chloride | | | | | | | | | | | |
| Sodium | 120.00 | 62.50 | 0.52 | 140 | 1.17 | | | | | | |
| phosphate | | | | | | | | | | | |
| monobasic•H₂O | | | | | | | | | | | |
| Calcium | 111.00 | 116.60 | 1.05 | 200 | 1.80 | 200 | 1.8018 | | | 33.22 | 0.30 |
| chloride, | | | | | | | | | | | |
| anhydrous | | | | | | | | | | | |
| Calcium | 236.00 | | | | | | | 100 | 0.42 | | |
| nitrate•4H₂O | | | | | | | | | | | |
| Sodium | 84.01 | 2438.0 | 29.02 | 2200 | 26.19 | 3700 | 44.0424 | 2000 | 23.81 | 1176 | 14.00 |
| bicarbonate | | | | | | | | | | | |
| Hepes buffer | | | | | | | | | | 142.04 | |
| (1M) | | | | | | | | | | | |

Trace Minerals

| | | | | | | | | | |
|---|---|---|---|---|---|---|---|---|---|
| Cupric | 249.70 | 0.0013 | 5.21E−06 | 0.0001 | 4E−07 | | | 0.0025 | 1.00E−05 |
| sulfate•5H₂O | | | | | | | | | |
| Ferrous | 278.00 | 0.42 | 1.50E−03 | | | | | 0.834 | 0.003 |
| sulfate•7H₂O | | | | | | | | | |
| Ferric | 101.10 | 0.05 | 4.95E−04 | 0.0001 | 9.9E−07 | 0.1 | 0.0010 | | |
| nitrate•9H₂O | | | | | | | | | |
| Zinc | 287.50 | 0.43 | 1.50E−03 | | | | | | |
| sulfate | | | | | | | | | |
| Zinc | | | | 0.0002 | | | | 0.863 | |
| sulfate•7H₂O | | | | | | | | | |
| Manganese | | | | 0.0001 | | | | | |
| chloride•4H₂O | | | | | | | | | |

Others

| | | | | | |
|---|---|---|---|---|---|
| Na | 2.39 | | | | 4.77 |
| hypoxanthine | | | | | |
| Phenol red | 8.10 | 10 | 15 | 5 | 1.2 |
| Glutathione | | 0.05 | | 1 | |
| (reduced) | | | | | |
| Methyl | | 0.03 | | | |
| linoleate | | | | | |

TABLE 3
Lineage stem cell supplement (50 mL units
for reconstitution in 1 L of basal media)

| | Formulation | |
|---|---|---|
| | (per 50 mL supplement or | |
| | 1 L of final media) | |
| Components | value | unit |
| Water | QS to 50 | ml |
| human serum albumin | 2.5 | g |
| Transferrin partially saturated | 20 | mg |
| Insulin | 20 | mg |
| T3 | 0.002 | mg |
| Selenite | 0.01 | mg |
| Progesterone | 0.005 | mg |
| Putrescine | 10 | mg |
| Catalase | 2.5 | mg |
| Glutathione | 1 | mg |
| Carnitine | 2 | mg |
| Biotin | 0.05 | g |
| L-glutamine | 365 | mg |
| Ethanolamine | 15 | mg |
| HEPES | 1 | g |
| Lipid Mix (see Table 4) | 5 | ml |

TABLE 4
Lipid mix

| | Concentration: |
|---|---|
| Components | μg/mLL |
| Linolenic | 10 |
| Linoleic | 10 |
| Tocopherol acetate | 50 |
| Cholesterol | 100 |

The lipid mix is made by o/w emulsions using Pluronic F68, phosphatidyl choline, Tween 80, cyclodextrin, or combinations thereof

In an alternative method, the activin/follistatin system can be used to isolate very early cancer stem cells. The addition of activin A can select a subpopulation of activin A-resistant CSC. Follistatin is used to block the activin A receptors and prevent spontaneous differentiation of the CSC, especially when large numbers of cells that endogenously secrete activin A are present, such as fibroblasts and normal cells. The use of follistatin has no effect if the cells are insensitive to activin A or in high purity CSC stem cell populations where follistatin can be secreted endogenously.

Activin A is a protein that is a member of the transforming growth factor-beta (TGF-beta) superfamily. When added or included in culture medium, activin helps maintain stem cell pluripotency and self-renewal. However, activin A promotes maturation and differentiation of young cells and cancer cells that are receptive. Therefore, an initial goal is in vitro fast expansion of the tumor that also sustains the proliferation of cancer stem cells by creating a proper autocrine environment in the culture. Although activin A may select a subpopulation of very young cancer stem cells, such conditions applied early in the manufacturing will greatly delay the expansion given the very low concentration of the cancer stem cells in the bulk. For example, a “fast expansion” is an expansion that results in the media in the culture vessels having obvious signs of consumption (change of pH for example) and the number of cells is visibly higher every day reflected by increased confluence.

For fast expansion, activin A is preferably omitted and not added, because it will slow down the culture growth. For some applications the interest is to obtain a very early stem cell population and the use of the activin A will select that cell population. Therefore, in one embodiment, an activin A-containing expansion is initiated and a first composition is administered to a subject comprising the activin A-activated cultured cells, followed by the isolation of the activin A-insensitive cells in an activin-A free culture and administering this second composition comprising the activin A free cultured cells to the subject.

In one embodiment, the media is supplemented with activin A at about 0.01 to 10 ng/mL, at about 0.05-9 ng/mL, at about 0.1-8 ng/mL, at about 0.5-7 ng/mL, at about 1-6 ng/mL, at about 1-5 ng/mL. In other embodiments, activin A is present in the media at about 0.5 ng/mL, at about 0.7 ng/mL, at about 0.9 ng/mL, at about 1 ng/mL, at about 1.25 ng/mL, at about 1.5 ng/mL, at about 1.75 ng/mL, at about 2 ng/mL, at about 2.25 ng/mL, at about 2.5 ng/mL, at about 2.75 ng/mL, at about 3 ng/mL, at about 3.5 ng/mL, at about 4 ng/mL, at about 4.5 ng/mL, at about 5 ng/mL, at about 6 ng/mL, at about 7 ng/mL, at about 8 ng/mL, at about 9 ng/mL, or at about 10 ng/mL.

Also disclosed is an embodiment wherein the media is supplemented with an antagonist of activin A, such as, but not limited to, follistatin or an antibody that specifically binds to activin A.

In another embodiment, the media is supplemented with follistatin at about 0.1 to 100 ng/mL, at about 0.5-50 ng/mL, at about 1-40 ng/mL, at about 2-30 ng/mL, at about 3-20 ng/mL, at about 5-15 ng/mL, at about 6-14 ng/mL, at about 7-13 ng/mL, at about 8-12 ng/mL, at about 9-11 ng/mL, or at about 10 ng/mL. In other embodiments, follistatin is present in the media at about 5 ng/mL, at about 6 ng/mL, at about 7 ng/mL, at about 8 ng/mL, at about 9 ng/mL, at about 11 ng/mL, at about 12 ng/mL, at about 12 ng/mL, at about 14 ng/mL, or at about 15 ng/mL.

In another embodiment, the media is supplemented with at least one ligand for a RTK. Ligands for RTKs are soluble or membrane-bound peptide/protein hormones including, but not limited to, NGF, PDGF, FGF, EGF, and insulin.

The combination of mitogens (e.g., FGF/EGF), activin A, and adherent substrate may result in an increase in the proliferation of normal cells such as fibroblasts or stellate cells. Thus, conditions are created to promote the expansion of very early CSC or progenitors that are insensitive to activin A in a rich environment or “stroma” constituted by cells with nourishing or encapsulating properties (e.g., fibroblasts, stellate cells). The colonies of CSC are progressively observed to develop along and spatially displace the stroma in the course of the next few days to weeks of cell culture. The media used in this method is the combination of the formulations described in Tables 2, 3 and 4.

There is a relationship between FGF, EGF, and activin A, and “very early” CSC. FGF and EGF cause proliferation of CSC in any differentiation status including the very early ones. Where activin A is in the cell culture medium, the activin A is permissive for (allows) proliferation exclusively of the very early CSC that are insensitive to activin A. If the CSC become sensitive, the proliferation will be stopped or reduced by activin A.

Insensitivity to FGF and EGF is not common and there are no natural blockers. Insensitivity to activin A can be mediated by follistatin, a natural blocker of the activin receptor. Follistatin can be secreted by the same tumor cell or by cells surrounding the tumor. Activin A is typically secreted by the cells surrounding the tumor, therefore it is possible that the expansion of the tumor is dependent on the surrounding cells (inhibiting) and by the tumor (promoting the expansion). The lack of receptor for activin A, a characteristic of the very early, undifferentiated cancer stem cells can prevent the control of the tumor by the surrounding tissue.

The in vitro cultures will contain embryonic stem cell-like colonies. These colonies may be surrounded by stromal cells, that can be normal fibroblasts, differentiated tumor cells, or mesenchymal transitioned tumor cells.

The present disclosure provides method for preparing VM-CSC where the total culturing time including time required for manipulations such as changing media, replating, centrifugation, and sedimenting, is less than five months, less than four months, less than three months, less than two months, less than one month, less than 150 days, less than 120 days, less than 90 days, less than 60 days, less than 30 days, or less than 150 days (+/−20 days), less than 120 days (+/−20 days), less than 90 days (+/−20 days), less than 60 days (+/−20 days), less than 30 days (+/−20 days). In exclusionary embodiments, the present disclosure can exclude any method for preparing cancer stem cells, and any population of cancer stem cells prepared by that method, where time required for manipulation is greater than one of the time-frames disclosed above. Also provided is a time in adherent culture that is indicated by one of the above time-frames. Also provided is a time in non-adherent culture that is one of the above time-frames. Moreover, provided is a combined time in adherent culture and in non-adherent culture that is identified by one of the above time-frames.

Epithelial to Mesenchymal Transition (EMT)

Tumors of epithelial origin are known to regress or trans-differentiate into a mesenchymal state. Epithelial phenotypes are immobile, contribute to volume growth of the tumor limited to the originating tissue and are typically more differentiated. When EMT occurs, the cells gain mobility and produce adjacent tissue infiltration and distant metastases. The transitioned cell also gains a stem cell-like phenotype, with the ability to replicate and differentiate resulting in a new tumor (metastasis) in the host tissue with characteristics of the originating (primary) tumor. By EMT, the tumor cells gain additionally immunosuppressive ability, drug pump and radioresistance.

The media composition and the physical selection method promote the EMT phenomenon in vitro. The advantage of using an EMT transitioned population as an immunogen is in prevention of tumor recurrences. The antigenicity of EMT cancer cells could enable the immune system to recognize and destroy mobile cancer cells that cause metastasis. In the process of metastasis these cells travel in very low number, seed the host tissue, revert to an epithelial phenotype (MTE transition), grow and form a new tumor that has similar characteristics with the primary tumor. The conditions necessary to cause in vitro EMT are spheroid formation in serum free media, stimulation with bFGF, stimulation with BMP2, 4, or 7, then plating on adherent substrate containing RGD (Arg-Gly-Asp) peptide motifs (e.g., collagen, gelatin, etc).

The EMT-CSC subpopulation is obtained by culturing CSC spheroids, early CSC, or mixed CSC under culture conditions as described in Table 1 and FIG. 1.

As used herein, the term “CSC” can generally refer to CSC spheroids, early CSC, mixed CSC, or EMT-CSC.

Expansion of CSC Cultures and Generation of VM-CSC

The CSC can be propagated and expanded indefinitely, as an additional characteristic of stem cells.

Furthermore, the CSC can be partial or totally differentiated. If the stem cell expansion conditions are removed, the CSC can slow down or stop the proliferation, and change morphology and phenotype to a more differentiated cell type. The morphology can become flat, epitheloid or stelate having multiple nuclei—a characteristic of the more mature cells or stelate cells.

The adherent cultures can be dissociated in single cell suspension and transferred to non-adherent (anti-biofouling) conditions to remove the anchorage dependent differentiated cells. After 2-3 days, the stem cells tend to aggregate and clonally expand in small spheroids that based on differential sedimentation can be separated from the single cells. The spheroids can be re-plated in adherent conditions and further propagated. This method will purify the culture stem cell content if the cultures are overtaken by differentiated cells or normal cells such as fibroblasts, from 1-30% to 90-99% stem cell content. The method can be repeated as many times needed in order to restore stem cell purity.

Small spheroids generally have the dimensions of between 0.1 mm and 2 mm. The size distribution, in terms of number of cells per small spheroid, is generally between 10 cells and 10,000 cells. The shape of a small spheroid can be spherical or oval, and can also occur as conglomerates of spherical or oval structures.

Vascular mimicry CSC are generated from CSC spheroids, early CSC, mixed CSC, or EMT-CSC by culturing the CSC is a defined medium on an adherent substrate, wherein the medium is supplemented with soluble laminin. Laminins are a family of glycoprotein heterotrimers composed of an alpha, beta, and gamma chains. To date, five alpha, four beta, and three gamma laminin chains have been identified that can combine to form 15 different isoforms. The laminin alpha-chains are considered to be the functionally important portion of the heterotrimers, as they exhibit tissue-specific distribution patterns and contain the major cell interaction sites.

Vascular endothelium expresses only two laminin isoforms, and their expression varies depending on the developmental stage, vessel type, and the activation state of the endothelium. Laminin 411 (composed of laminin alpha4, beta1, and gamma1 chains) is expressed by all endothelial cells regardless of their stage of development, and its expression is strongly upregulated by cytokines and growth factors that play a role in inflammatory events. Laminin 511 (composed of laminin alpha 5, beta 1, and gamma 1 chains) is detectable primarily in endothelial cell basement membranes of capillaries and venules commencing 3-4 weeks after birth.

Laminin 211 (laminin-2 or merosin) is composed of alpha2, beta1, and gamma1 chains and is produced by myocytes in heart, and skeletal and smooth muscle tissues. In addition, constitutive expression of Laminin 211 is found in mouse and human thymic tissue vital for lymphoepithelial interactions.

Investigations in the tumoral secretion of laminins revealed that stromal and parenchymal vascularity was associated with laminin alpha2 chain expression in supraglottic carcinomas, whereas in other tumours, laminin alpha2 chain-positive vessels were observed only in the stroma.

Laminin 521 contains alpha5, beta2, and gamma1 chains and is normally expressed and secreted by human pluripotent embryonic stem cells and can, in addition, also be found in the kidneys, neuromuscular junctions, lungs and placenta.

Immunohistochemical studies of glioma tumor tissue demonstrated expression of alpha2, alpha3, alpha4 and alpha5, but not alpha1, laminins by the tumor vasculature. The results indicate that glioma cells secrete alpha2, alpha4 and alpha5 laminins and that alpha3 and alpha5 laminins, found in brain vasculature, selectively promote glioma cell migration.

In one embodiment, the laminin is a soluble laminin comprising at least an alpha1, alpha2, alpha3, or alpha4 chain. In another embodiment, the laminin is in a monomer, dimer, or trimer form. The laminin is added to the media n solution to the cell cultures, not as a substrate. Furthermore, the laminin is not an insoluble polymer form.

A patient-specific VM-CSC cell line can be used to identify the genomic mutation responsible for the neoplastic transformation when compared with normal tissue from the same patient. The genomic mutation may not be expressed in every stage of differentiation. Some regulatory proteins, or transcription factors, are only temporary expressed and may disappear during maturation, resulting in a malformed cell but with normal proteins. Identification of a cell population that is maximally expressing the mutation and exposing this population to the immune system could be a major advantage of using cancer stem cells as an antigen source for immune-therapy.

By identifying the genomic mutation a personalized formulation can be created for a cancer treatment, for example a small molecule, a DNA sequence, antisense RNA or combinations.

Such cell lines can be further used to create screening plates (96 wells for example) for drug discovery. Multiple lines from various patients can be combined in a single plate to address variability between individuals.

Vascular mimicry carcinoma cancer stem cells may retain some properties of the originating tissue such as secretion of proteins, growth factors and hormones (functional tumors). These properties can be exploited given the immortal characteristics of the cell lines, to produce “self” proteins that can be used for the same patients (for example albumin, transforming growth factor (TGF), insulin, glucagon, DOPA etc). The cells can be introduced in small bioreactors and the secretion product collected, purified and stored for the same patient use. This method is particularly advantageous that the patient will not develop immune resistance such as the more traditional biosimilars.

Loading Dendritic Cells

The individual VM-CSC cells obtained from the patient can be used to produce an antigen for immune therapy. The advantage of using the purified stem cell line resides in a better signal to noise ratio. The more mature cells from the tumor may have compensatory mechanisms that can mask the antigenicity and could be not identified by the immune system. As an antigen source, the VM-CSC can be used alive, mitotically inactive, nonviable or fragmented. Various methods can be used to modify the cells for optimal antigen exposure: a radiant energy (e.g., gamma, UV, X), temperature (e.g., heat or cold), or chemical (e.g., cytostatic, aldehyde, alcohol) or combinations.

In exemplary implementations, the present disclosure encompasses reagents and methods for activating dendritic cells (DCs), with one or more immune adjuvants, such as GM-CSF, a toll-like receptor (TLR) agonist, e.g., CpG-oligonucleotide (TLR9), imiquimod (TLR7), poly(I:C) (TLR3), glucopyranosyl lipid A (TLR4), murein (TLR2), flagellin (TLR5), as well as an adjuvant such as CD40 agonists, e.g., CD40-ligand, or the cytokine, interferon-gamma, prostaglandin E2, and the like. See, e.g., U.S. Pat. No. 7,993,659; U.S. Pat. No. 7,993,648; U.S. Pat. No. 7,935,804, each of which is incorporated herein by reference for all it discloses regarding activating DCs. The present disclosure encompasses ex vivo treatment of DCs with one or more of the above adjuvant reagents, or in addition, or alternatively, administration of the adjuvant to a human subject, animal subject, or veterinary subject.

The immune system encompasses cellular immunity, humoral immunity, and complement response. Cellular immunity includes a network of cells and events involving dendritic cells, CD8⁺ T cells (cytotoxic T cells; cytotoxic lymphocytes), and CD4⁺ T cells (helper T cells). Dendritic cells (DCs) acquire polypeptide antigens, where these antigens can be acquired from outside of the DC, or biosynthesized inside of the DC by an infecting organism. The DC processes the polypeptide, resulting in peptides of about ten amino acids in length, transfers the peptides to either MHC class I or MHC class II to form a complex, and shuttles the complex to the surface of the DC. When a DC bearing a MHC class I/peptide complex contacts a CD8⁺ T cell, the result is activation and proliferation of the CD8⁺ T cell. Regarding the role of MHC class II, when a DC bearing a MHC class II/peptide complex contacts a CD4⁺ T cell, the outcome is activation and proliferation of the CD4⁺ T cell. Although dendritic cells presenting antigen to a T cell can “activate” that T cell, the activated T cell might not be capable of mounting an effective immune response. Effective immune response by the CD8⁺ T cell often requires prior stimulation of the DC by one or more of a number of interactions. These interactions include direct contact of a CD4⁺ T cell to the DC (by way of contact the CD4⁺ T cell's CD40 ligand to the DCs CD40 receptor), or direct contact of a TLR agonist to one of the dendritic cell's toll-like receptors (TLRs).

Humoral immunity refers to B cells and antibodies. B cells become transformed to plasma cells, and the plasma cells express and secrete antibodies. Naïve B cells are distinguished in that they do not express the marker CD27, while antigen-specific B cells do express CD27. The secreted antibodies can subsequently bind to tumor antigens residing on the surface of tumor cells. The result is that the infected cells or tumor cells become tagged with the antibody. With binding of the antibody to the infected cell or tumor cell, the bound antibody mediates killing of the infected cell or tumor cell, where killing is by NK cells. Although NK cells are not configured to recognize specific target antigens, in the way that T cells are configured to recognize target antigens, the ability of NK cells to bind to the constant region of antibodies, enables NK cells to specifically kill the cells that are tagged with antibodies. The NK cell's recognition of the antibodies is mediated by Fc receptor (of the NK cell) binding to the Fc portion of the antibody. This type of killing is called, antibody-dependent cell cytotoxicity (ADCC). NK cells can also kill cells independent of the mechanism of ADCC, where this killing requires expression of MHC class I to be lost or deficient in the target cell.

Without wishing to be bound to any particular mechanism, the disclosure encompasses administration of cancer stem cell antigens, or administering dendritic cells loaded with cancer stem cell antigens, where the antigens stimulate the production of antibodies that specifically recognize one or more of the cancer stem cell antigens, and where the antibodies mediate ADCC. The phrase “loaded with antigens” refers to the ability of the dendritic cell to capture live cells, to capture necrotic cells, to capture dead cells, to capture polypeptides, or to capture peptides, and the like. The tumor antigens comprise cell extracts of the VM-CSC, lysates of the VM-CSC, or intact VM-CSC cells. In another embodiment, the tumor antigens comprise messenger RNA transfected into the dendritic cells ex vivo.

Capture by cross-presentation is encompassed by the present disclosure. Also encompassed is the use of antigen-presenting cells that are not dendritic cells, such as macrophages or B cells.

The technique of “delayed type hypersensitivity response” can be used to distinguish between immune responses that mainly involve cellular immunity or mainly involve humoral immunity. A positive signal from the delayed type hypersensitivity response indicates a cellular response.

The present disclosure provides compositions and methods, where tumor cells are inactivated, e.g., by radiation, nucleic acid cross-linkers, polypeptide linkers, or combinations of these. Cross-linking is the attachment of two chains of polymers molecules by bridges, composed of either an element, a group, or a compound that join certain carbon atoms of the chains by primary chemical bonds. Cross-linking occurs in nature in substances made up of polypeptide chains that are joined by the disulfide bonds involving two cysteine residues, as in keratins or insulin, trivalent pyridinoline and pyrrole cross-links of mature collagen, and cross-links in blood clots which involve covalent epsilon-(gamma-glutamyl)lysine cross-links between the gamma-carboxy-amine group of a glutamine residue and the epsilon-amino group of a lysine residue.

Cross-linking can be artificially effected in proteins, either adding a chemical substance (cross-linking agent), or by subjecting the polymer to high-energy radiation. Cross-linking with fixatives and heat-induced aggregation has been shown to enhance immune responses as well as completely inhibit proliferation. Substances that may be used to cross-link proteins on the surface, and therefore prevent proliferation, of VM-CSC include, but are not limited to, 10% neutral-buffer formalin, 4% paraformaldehyde, 1% glutaraldehyde, 0.25-5 mM dimethyl suberimidate, ice-cold 100% acetone or 100% methanol. Additionally, combinations of 1% glutaraldehyde and 4% paraformaldehyde in 0.1 M phosphate buffer solution may also be used.

Formaldehyde and glutaraldehyde have both been shown to induce the activation of T helper type 1 and type 2 cells. In particular, heat induced aggregation of antigens was also shown to enhance the in vivo priming of cytotoxic T lymphocytes. Cross-linking of antigens by 3,3′-dithiobis(sulfosuccinimidylpropionate) results in increased binding of antigens to dendritic cells and the cross-linked antigens are processed through the proteosomal pathway for antigen presentation. Furthermore, formalin fixed tumor cells have been used in clinical trials with no evidence of proliferation.

In one embodiment, whole VM-CSC are fixed with cross-linking agents, and then used as the antigen source in combination with the dendritic cells.

In another embodiment, the nucleic acids of the cells are cross-linked. An exemplary nucleic acid alkylator is beta-alanine, N-(acridin-9-yl), 2-[bis(2-chloroethyl)amino]ethyl ester. Exemplary cross-linkers, such as psoralens, often in combination with ultraviolet (UVA) irradiation, have the ability to cross-link DNA but to leave proteins unmodified. For instance, the nucleic acid targeting compound can be 4′-(4-amino-2-oxa)butyl-4,5′,8-trimethylpsoralen (S-59). Cells can be inactivated with 150 μM psoralen S-59 and 3 J/cm² UVA light (FX 1019 irradiation device, Baxter Fenwal, Round Lake, Ill.). The inactivation with S-59 with UV light is referred to as photochemical treatment, where treatment conditions can be adjusted or titrated to cross-linked DNA to the extent that cell division is completely prevented, but where damage to polypeptides, including polypeptide antigens, is minimized. Cells can be suspended in 5 mL of saline containing 0, 1, 10, 100, and 1000 nM of psoralen S-59. Samples can be UVA irradiated at a dose of approximately 2 J/cm². Each sample can then transferred to a 15 mL tube, centrifuged, and the supernatant removed, and then washed with 5 mL saline, centrifuged and the supernatant removed and the final pellet suspended in 0.5 mL of saline. See U.S. Pat. Nos. 7,833,775 and 7,691,393, which are incorporated herein by reference for all they disclose regarding inactivation of cells.

For any cell preparation that is treated with a cross-linking agent, the ability to divide can be tested by the skilled artisan by incubating or culturing in a standard medium for at least one week, at least two weeks, at least three weeks, at least four weeks, at least five weeks, at least two months, at least three months, at least four months, and so on. Cell division can be assessed by stains that reveal chromosomes, and that reveal that cell division is, or is not, taking place. Cell division can also be measured by counting cells. Thus, where the number of cells in a culture plate remains stable for a period of two weeks, one month, or two months, and so on, it can reasonably be concluded that the cells cannot divide.

In one embodiment, the dendritic cell immunogenic composition is administered subcutaneously (SC). In further embodiments, each dose ranges from about 5-20 million loaded DCs, repeated in a series of 6-10 doses. In certain embodiments, the doses are administered every five days, every week, every 10 days, every other week, or every third week for two, three, four, five or six doses, followed by administration of doses every two weeks, every three weeks, every four weeks, every month, every five weeks, or every 6 weeks for two, three, four, five or six doses additional doses for a total of 6-10 doses. In one embodiment, the first four injections are given every week for a month, and then once a month for the next 4 injections. In alternative embodiment, administration is once a week for 3 weeks then once a month for 5 months for a total of 8 administrations.

Each dose comprises about 5-20×10⁶ loaded DCs, about 5-17×10⁶ loaded DCs, about 6-16×10⁶ loaded DCs, about 7-15×10⁶ loaded DCs, about 7-14×10⁶ loaded DCs, about 8-13×10⁶ loaded DCs, about 8-12×10⁶ loaded DCs, or about 9-11×10⁶ loaded DCs. In additional embodiment, each dose comprises about 8×10⁶ loaded DCs, about 9×10⁶ loaded DCs, about 10×10⁶ loaded DCs, about 11×10⁶ loaded DCs, or about 12×10⁶ loaded DCs. The loaded DCs comprise a mixture of DCs and residual VM-CSCs which have not been taken up by the DCs. The administered dose comprises a mixture of these cells and the dose reflects this mixture.

In another embodiment, the loaded DCs are administered with a pharmaceutically acceptable carrier or excipients. The pharmaceutically acceptable excipients described herein, for example, vehicles, adjuvants, carriers or diluents, are well-known to those who are skilled in the art and are readily available to the public. It is preferred that the pharmaceutically acceptable carrier or excipient be one which is chemically inert to the loaded DCs and one which has no detrimental side effects or toxicity under the conditions of use.

The choice of excipient or carrier will be determined in part by the particular therapeutic composition, as well as by the particular method used to administer the composition. The formulations described herein are merely exemplary and are in no way limiting.

Often the physiologically acceptable carrier is an aqueous pH buffered solution. Examples of physiologically acceptable carriers include, but are not limited to, saline, solvents, dispersion media, cell culture media, aqueous buffers such as phosphate, citrate, and other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

In some exemplary implementations, an adjuvant is given simultaneously with every dose. In certain embodiments, the cell dose is suspended in a carrier containing an adjuvant. In alternative exemplary implementations, an adjuvant is administered, but not with every single dose. In other exemplary implementations, there is no adjuvant at all. In one embodiment, the adjuvant is GM-CSF.

Without limitation, dendritic cells (e.g., autologous or allogeneic dendritic cells) are contacted with cancer stem cell antigens as a cell lysate, acid elution, cell extract, partially purified antigens, purified antigens, isolated antigens, partially purified peptides, purified peptides, isolated peptides, synthetic peptides, or any combination thereof. The dendritic cells are then administered to a subject, for example, a subject having cancer, or a control subject not having cancer. In exemplary implementations, dendritic cells are contacted with, injected into, or administered, by one or more of a route that is subcutaneous, intraperitoneal, intranodal, intramuscular, intravenous, intranasal, inhaled, oral, by application to intestinal lumen, and the like. Additionally, the immunogenic compositions can be administered directly to the site of a tumor or metastasis.

### EXAMPLES

### Example 1

### MATRIGEL®-Based Tube Formation Assay for Testing of Vasculogenic Properties of Cells

A tube formation assay using growth factor-reduced thick layer of MATRIGEL® (BD Biosciences) has been employed to demonstrate the angiogenic activity of vascular endothelial cells in vitro. MATRIGEL® is a reconstituted basement membrane isolated from the Engelbreth-Holm-Swarm (EHS) mouse sarcoma and therefore not suitable for growth of human cells for clinical use. MATRIGEL® has many constituents, predominantly composed of laminin 111, collagen IV, entactin, and heparin sulfate proteoglycan. This assay can be used to test the ability of a number of tumor cells to develop a vascular phenotype or vascular mimicry. Vascular mimicry (VM) is a process by which vasculogenic-like networks are formed by tumor cells, specifically, cancer stem cells (CSCs), rather than by epithelial cells and these cells mimic the process of vasculogenesis.

Commercially available cancer lines SK-MEL-28, SK-MEL-5, and two patient patient-derived melanoma cancer stem cell lines were subjected to both VM-inducing and non-VM inducing conditions.

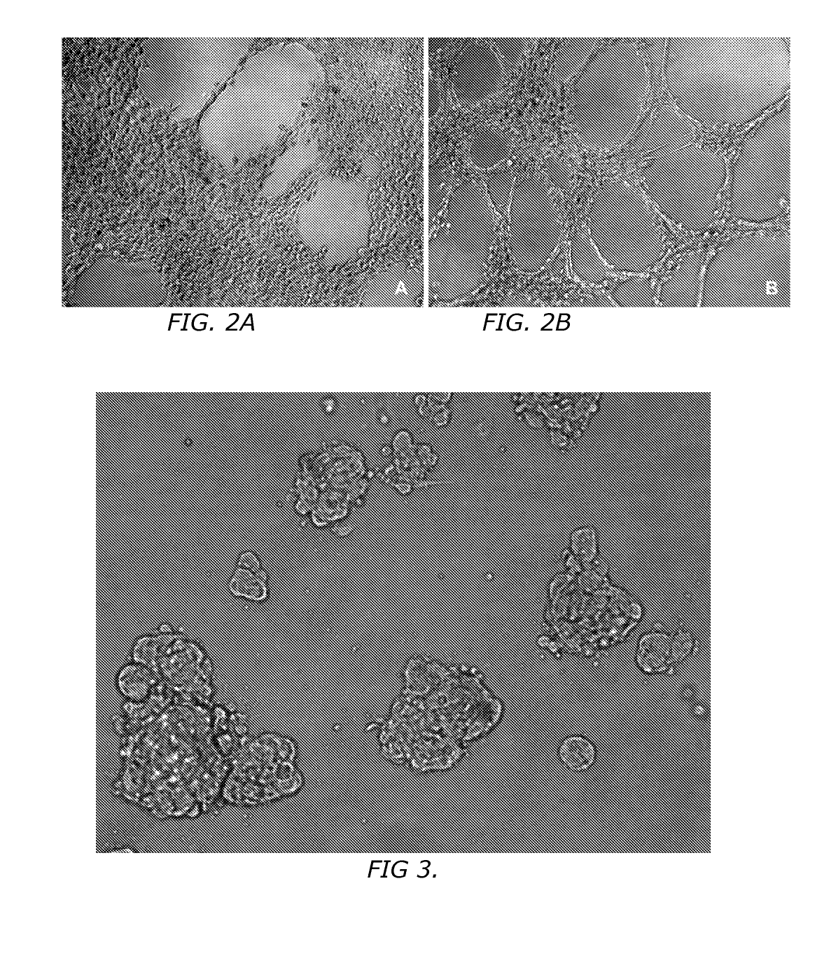

Traditional thick layer MATRIGEL®-based protocol was employed as a VM-inducing condition. Growth factor-reduced MATRIGEL® was warmed up at room temperature and transferred on ice for 10 minutes. Fifty microliters of MATRIGEL® were plated to each well of 96-well plate at a horizontal level and MATRIGEL® was allowed to distribute and incubate for 30 min at 37° C. Tumor cells (1-10×10⁴) were re-suspended with serum-free DMEM media and loaded on top of the MATRIGEL® and allowed to incubate for 24-72 hours. Vascular network formation was typically observed after 24 hours in the commercial lines (FIG. 2A) and patient-derived lines (FIG. 2B).

Diluted, thin-layered MATRIGEL® was used as a non-VM-inducing condition. Growth factor-reduced MATRIGEL® was warmed up at room temperature and diluted 1:30 or 1:60 with serum-free DMEM media. Fifty microliters of MATRIGEL® were plated to each well of 96-well plate at a horizontal level and MATRIGEL® was allowed to distribute and incubate for 2 hours at 37° C., after which MATRIGEL® was aspirated. Tumor cells (1-10×10⁴) were re-suspended with serum-free DMEM media and loaded on top of thin-layered MATRIGEL® and allowed to incubate for 24-72 hours. While thin-layered MATRIGEL® is commonly used as a cell substrate, it does not provide the environment needed for VM formation in the cells that possess the VM phenotype, the cells expanded as a monolayer.

Patient-derived melanoma cancer stem cell lines have demonstrated the ability to form vascular network in the previously described VM-inducing condition, similar to commercially available cell lines.

### Example 2

The following experiment was designed to define conditions to obtain a high purity population of cancer stem cells that is responsible for VM or presents a VM transformation that can provide a source for antigen for immunotherapy using methods that are relevant for clinical manufacturing.

Cancer stem cells derived from a patient-sourced melanoma sample were exposed to a laminin with an alpha2 chain (211), a laminin with a alpha5 chain (521), and to a common laminin used in cell culture, that contain mostly an alpha1 chain (mouse sarcoma extract that is mostly made of laminin-111). As MATRIGEL® is recognized as a standard test for vascular transformation of the tumor cells, it was used in a low concentration, high concentration, or thin coating.

Recombinant human laminins 211 and 521 were used in a concentration of 10μ/mL; EHS laminin (111) was used in concentrations of 10 and 50 μg/mL and MATRIGEL® in a dilution of 1:60 and 1:4. The reagents were added to a serum-free DMEM or CSC-M media (Table 5) to test the induction of VM on patient melanoma lines.

TABLE 5
Media formulations used in purification and expansion of CSC

| Media | Description | Component | Concentration | |
|---|---|---|---|---|
| CSC-M | CSC Tumor Microsphere | DMEM:F12 | 450 | mL |
| | generation media | (Table 2) | | |
| | (selection media) | Lineage supplement | 50 | mL |
| | | (Table 3) | | |
| CSC-E | CSC Tumor Expansion | CSC-M | 85% | v/v |
| | media | FBS | 15% | v/v |

Patient-derived tumor cells were obtained from bulk tumors that were digested in collagenase I, exposed to a serum free media (CSC-M) in the presence of 10 ng/mL bFGF and 10 ng/mL EGF in a ultra-low adherent cell culture flask to select and expand spheroids, and finally plated and expanded in a regular tissue culture flask in CSC-E media\. (see WO 2014/028274 which is incorporated herein for all it discloses regarding cancer stem cell culture media).

The cells obtained as described above, were distributed in 300 μL media containing soluble laminins or MATRIGEL® and placed in each well of 96-well plate as detailed in Table 6. Cells were grown for 24-72 hours. Early evidence of vascular networks and VM-induced markers was observed after 24 hours.

TABLE 6
Experimental conditions

| | Media condition | Media condition |
|---|---|---|
| | DMEM | CSC-M |
| VM Inducer | Laminin 211 (10 μg/mL) | Laminin 211 (10 μg/mL) |
| condition | Laminin 521 (10 μg/mL) | Laminin 521 (10 μg/mL) |
| | Laminin EHS (10 μg/mL) | Laminin EHS (10 μg/mL) |
| | Laminin EHS (50 μg/mL) | Laminin EHS (50 μg/mL) |
| | MATRIGEL ® 1:60 | MATRIGEL ® 1:60 |
| | MATRIGEL ® 1:4 | MATRIGEL ® 1:4 |
| | MATRIGEL ® thin layer coating | MATRIGEL ® thin layer coating |

After 72 hours, the cells were fixed in 4% paraformaldehyde and stained for VEGF-R2 (vascular endothelial growth factor receptor 2) VEGF-R1 (vascular endothelial growth factor receptor 1), VE-Cadherin (vascular endothelial cadherin, CD144), VEGF-A (vascular endothelial growth factor A), CD34, vWF (von Willebrand Factor), PECAM (platelet endothelial cell adhesion molecule, CD31) and UEA-I (Ulex europaeus agglutinin I).

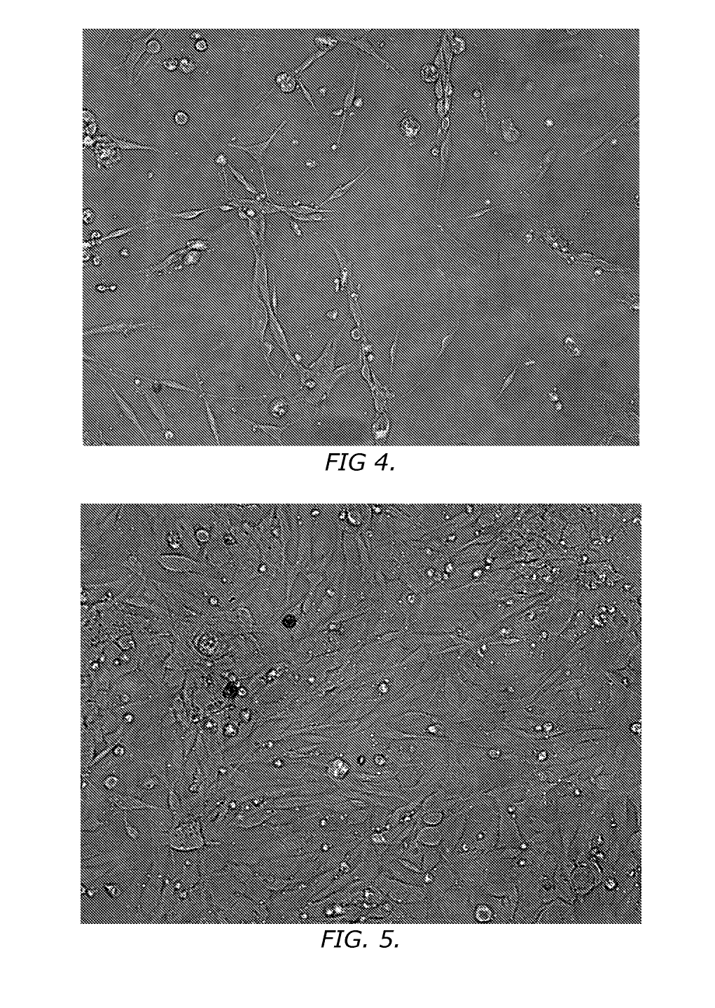

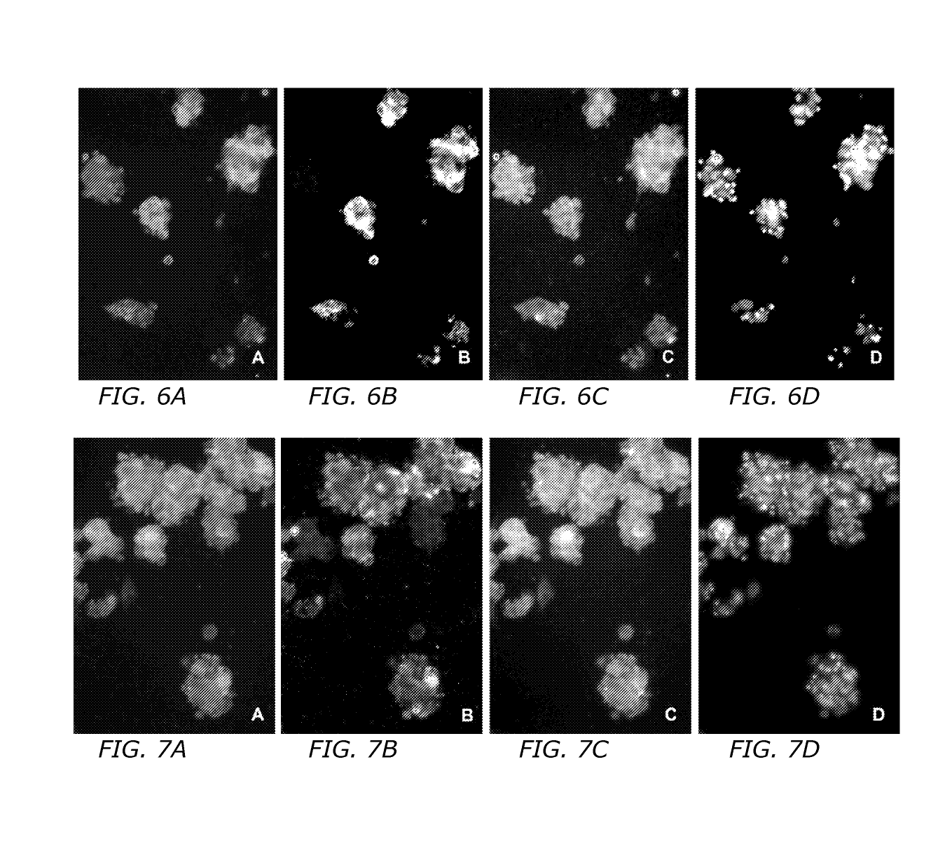

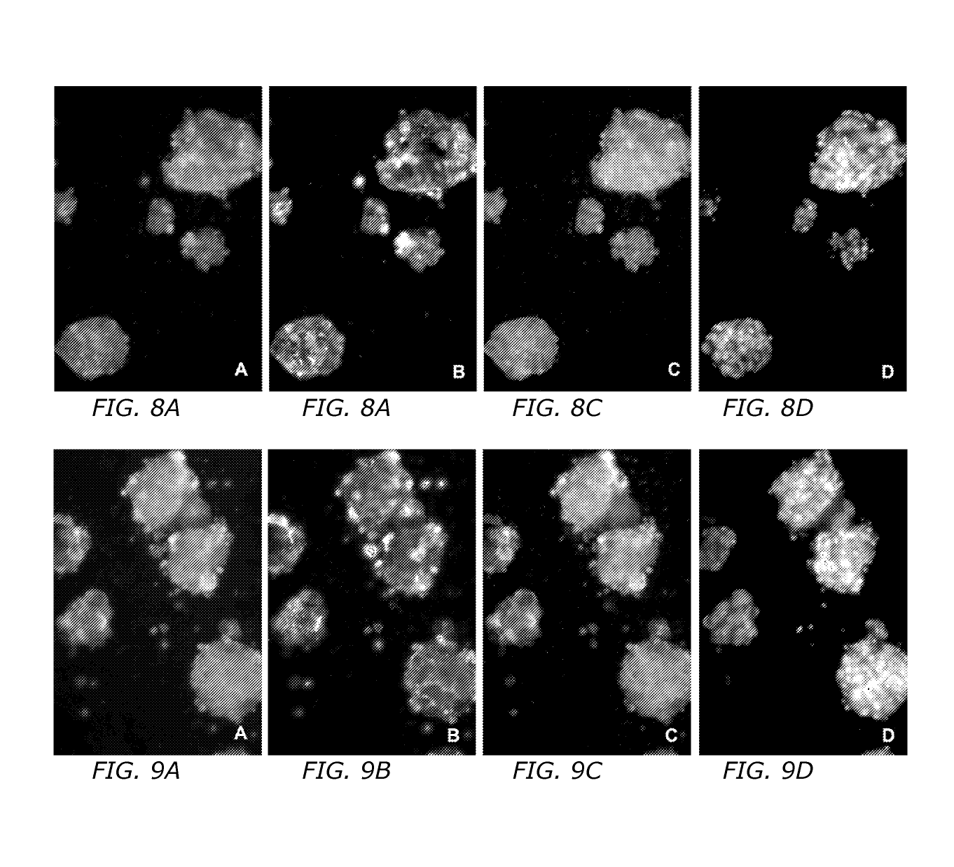

The evidence of vascular transformation is morphologically reflected by appearance of typical strings (FIG. 4). In some conditions buds (FIG. 3) or conglomerates of cells that adhere to each other can be observed along with the strings. A non-vascular feature is the expansion of cells as a monolayer (FIG. 5). Each of these elements was quantitated with scores between 0 and 3, where 0=absence of the specification and 3 is abundance of the specification. The results are summarized in Table 7.

TABLE 7
Quantification of the morphology of the cell growth in various conditions

| | DMEM | | | CSC-M | | |
|---|---|---|---|---|---|---|
| Condition/Morphology | Buds | Strings | Monolayer | Buds | Strings | Monolayer |
| Laminin 211 (10 μg/mL) | 3 | 2 | 2 | 3 | 0 | 0 |
| Laminin 521 (10 μg/mL) | 0 | 1 | 3 | 1 | 0 | 3 |
| Laminin EHS (10 μg/mL) | 0 | 1 | 3 | 3 | 0 | 0 |
| Laminin EHS (50 μg/mL) | 3 | 1 | 0 | 3 | 2 | 1 |
| MATRIGEL ® 1:60 | 1 | 2 | 1 | 3 | 1 | 0 |
| MATRIGEL ® 1:4 | 1 | 2 | 0 | 1 | 2 | 0 |
| MATRIGEL ® pre-coated | 0 | 0 | 3 | 0 | 0 | 3 |

Scoring: 3 = abundant occurrence; 2 = some occurrence; 1 = few occurrence; 0 = Absent

Buds and strings that are the morphological signature of VM were observed in the conditions containing laminin 211, and soluble MATRIGEL®. The laminin 111 (EHS) condition produced buds and strings only at higher concentration in DMEM while in low concentration only in CSC-M media. Thin MATRIGEL® coating and laminin 521 produced exclusively monolayers, regardless of media composition. Concentrated MATRIGEL® produced strings as described in literature.

Considering that the bud and string formation are reflecting a vasculogenic mimicry (VM), in the Table 8 the total scores for each media condition is summarized and in Table 9 the total scores for each reagent condition are presented.

TABLE 8
Summary of the VM and Non-VM scores for each media condition

| | Morphology | | | |
|---|---|---|---|---|
| | DMEM | | CSC-M | |
| | Total | | Total | |
| Condition | VM | Non-VM | VM | Non-VM |
| Laminin 2 (10 μg/mL) | 5 | 2 | 3 | 0 |
| Laminin 11 (10 μg/mL) | 1 | 3 | 1 | 3 |
| Laminin EHS (10 μg/mL) | 1 | 3 | 3 | 0 |
| Laminin EHS (50 μg/mL) | 4 | 0 | 5 | 1 |
| MATRIGEL ® 1:60 | 3 | 1 | 4 | 0 |
| MATRIGEL ® 1:4 | 3 | 0 | 3 | 0 |
| MATRIGEL ® pre-coated | 0 | 3 | 0 | 3 |
| Sum of scores | 18 | 12 | 19 | 7 |
| Score percentage | 60% | 40% | 73% | 27% |

As shown in the Table 9, the media composition can influence the preponderance of the vascular transformation. In comparison with the typical literature described method that uses a simple DMEM media, by using a richer composition (CSC-M) the percentage of vascular transformation increased to 73% from 60% across all conditions. The media formulation alone is not sufficient to cause the VM transformation, as the thin-coated MATRIGEL® condition produced exclusively monolayers as expected.

TABLE 9
Summary of the VM and Non-VM scores for
each inducer in both media conditions

Morphology

| | Total | Total | % VM |
|---|---|---|---|
| Condition | VM | non-VM | morphology |
| Laminin 211 (10 μg/mL) | 8 | 2 | 80 |
| Laminin 521 (10 μg/mL) | 2 | 6 | 25 |
| Laminin EHS (10 μg/mL) | 4 | 3 | 57 |
| Laminin EHS (50 μg/mL) | 9 | 1 | 90 |
| MATRIGEL ® 1:60 | 7 | 1 | 87 |
| MATRIGEL ® 1:4 | 6 | 0 | 100 |
| MATRIGEL ® pre-coated | 0 | 6 | 0 |

As shown in Table 8 the presence of a particular laminin in solution has a dramatic effect on the vascular morphology induction. As expected the concentrated MATRIGEL® produced exclusively VM, followed closely by laminin 211 (80%) and laminin 111 (90%) in high concentration. Low concentration of laminin 111 or the tested concentration of laminin 511 produced significantly lower amounts of VM morphology (57% and 25%).

The immunocytochemical (ICC) analysis of the conditions revealed that the typical vascular endothelial markers are correlating with the morphological observations. The results are summarized in Table 10 for the DMEM media and Table 11 for the CSC-M media.

TABLE 10
ICC characterization of the cultures grown in DMEM media

| Condition/Marker | VEGF-R2 | VEGF-R1 | VE-Cadh | VEGF-A | CD34 | VWF | PE-CAM | UEA-I |
|---|---|---|---|---|---|---|---|---|
| Laminin 211 (10 μg/mL) | +++ 4 | +++ 4 | + 4 | ++ 4 | ++ 4 | +++ 4 | ++ 4 | +++ 3 |
| Laminin 521 (10 μg/mL) | −− 0 | +++ 4 | + 4 | ++ 4 | ++ 4 | +++ 4 | + 3 | +++ 2 |
| Laminin EHS (10 μg/mL) | −− 0 | ++ 2 | + 3 | ++ 1 | ++ 1 | +++ 2 | + 2 | +++ 1 |
| Laminin EHS (50 μg/mL) | + 1 | +++ 4 | ++ 3 | ++ 2 | ++ 3 | +++ 4 | ++ 3 | +++ 3 |
| MATRIGEL ® 1:60 | ++ 3 | +++ 3 | + 2 | +++ 1 | ++ 4 | +++ 3 | + 1 | −− 0 |
| MATRIGEL ® 1:4 | −− 0 | + 1 | −− 0 | −− 0 | ++ 3 | + 2 | −− 0 | + 1 |
| MATRIGEL ® pre-coated | + 1 | +++ 3 | + 1 | ++ 2 | + 1 | +++ 3 | −− 0 | +++ 2 |

Staining intensity scale: −− absent; + low; ++ medium; +++ high intensity.
Percentage of positive cells estimation: 0 = absent; 1 = 1-24%; 2 = 25%-49%; 3 = 50%-75%; 4 = 75% 100% from total cells in the microscope field.

TABLE 11
ICC characterization of the cultures grown in CSC-M media

| Condition/Marker | VEGF-R2 | VEGF-R1 | VE-Cadh | VEGF-A | CD34 | VWF | PE-CAM | UEA-I |
|---|---|---|---|---|---|---|---|---|
| Laminin 211 (10 μg/mL) | +++ 4 | +++ 4 | ++ 4 | +++ 4 | +++ 4 | +++ 4 | +++ 4 | +++ 4 |
| Laminin 521 (10 μg/mL) | −− 0 | +++ 3 | + 2 | +++ 2 | +++ 2 | +++ 3 | +++ 3 | +++ 2 |
| Laminin EHS (10 μg/mL) | ++ 4 | +++ 4 | ++ 4 | +++ 2 | +++ 3 | +++ 3 | +++ 4 | +++ 3 |
| Laminin EHS (50 μg/mL) | + 4 | +++ 4 | ++ 3 | ++ 3 | +++ 4 | +++ 4 | ++ 4 | +++ 4 |
| MATRIGEL ® 1:60 | ++ 3 | +++ 2 | ++ 4 | + 1 | +++ 4 | +++ 4 | ++ 4 | ++ 1 |
| MATRIGEL ® 1:4 | ++ 4 | +++ 2 | +++ 4 | ++ 1 | +++ 4 | +++ 4 | +++ 4 | +++ 4 |
| MATRIGEL ® pre-coated | + 1 | +++ 2 | + 3 | +++ 1 | + 2 | +++ 3 | ++ 1 | +++ 1 |

Staining intensity scale: −− absent; + low; ++ medium; +++ high intensity.
Percentage of positive cells estimation: 0 = absent; 1 = 1-24%; 2 = 25%-49%; 3 = 50%-75%; 4 = 75% 100% from total cells in the microscope field.

To create a general overview of the VM profile, the scores of each marker was summed across the same inducer condition and expressed as a percentage of the maximum possible score, as if the cells were 100% expressing all the VM markers. The results are reproduced in Table 12.

TABLE 12
Summary of the VM-ICC profile scores in two media formulations

| | Media | | | |
|---|---|---|---|---|
| | DMEM | | CSC-E | |
| | Sum of | | Sum of | |
| Condition | VM scores | % | VM score | % |
| Laminin 211 (10 μg/mL) | 31 | 97 | 32 | 100 |
| Laminin 521 (10 μg/mL) | 25 | 78 | 17 | 53 |
| Laminin EHS (10 μg/mL) | 13 | 41 | 23 | 72 |
| Laminin EHS (50 μg/mL) | 23 | 72 | 30 | 94 |
| MATRIGEL ® 1:60 | 17 | 53 | 23 | 72 |
| MATRIGEL ® 1:4 | 7 | 21 | 27 | 84 |
| MATRIGEL ® thin layer | 13 | 42 | 14 | 44 |

DMEM is a simple media composition that does not sustain the proper metabolic needs of living cells. For that reason, the VM markers in this condition do not reflect the morphological changes observed after 72 hours of incubation. In particular, the MATRIGEL® conditions are surprisingly low in the number of VM positive cells. In this case, it was possible that a distress mechanism in combination with the extracellular matrix could induce the VM morphology. In a more complex media composition (CSC-M) the positivity for VM markers perfectly correlates with the observed morphology: the most VM generating conditions are laminin 211 (100%), laminin 111 at high concentration (94%) and MATRIGEL® in high concentration (84%).

Interestingly in the conditions were cultures did not displayed the typical VM morphology, and expanded in a monolayer format (MATRIGEL® thin layer). A good proportion of these cells displayed the presence of VM markers (42-44%). Due to the lack of CSC maintenance and propagation conditions (5% FBS, FGF, EGF), the cells spontaneously display a propensity for the VM phenotype.

A VM phenotype can be reliably produced in patient-derived cancer cell lines as follows: 1) isolate and expand cancer stem cells from the tumor bulk; and 2) expose the cultures to a soluble laminin, preferable containing alpha2 chain, or alpha1 chain in higher concentration. If the cultures are exposed to a solution of laminin alpha5, or low concentration of alpha1, the CSC cultures can be further expanded as a monolayer in a reach media composition without significant VM transformation. The domain structure of alpha4 is similar to that of alpha3, both of which resemble truncated versions of alpha1 and alpha2 chains, and given that the laminin activity is mainly attributed to the alpha chain, a VM-inducing effect of the alpha3 and alpha4 laminins on the cancer stem cells may be seen.

### Example 3

### Production of Loaded Dendritic Cell Compositions

The antigen source is autologous tumor cells from continuously proliferating, self-renewing cells derived from the patient's fresh tumor tissue. These cells have the characteristics of tumor stem cells. At all times in the surgical and pathology setting, biopsies are handled with strict adherence to sterility protocols to ensure that samples are sterile.

The pathologist obtains fresh tissue from biopsy of the patient's tumor. Using sterile scalpels and forceps, the specimen is cut into 10 mm slices and transferred to the transport tubes containing transport media, working quickly to avoid specimen drying. Specimens are shipped by overnight courier to the manufacturing facility within 48 hours of surgical resection.

At the manufacturing facility, samples are dissociated into single cell suspensions in a clean room and placed in cell culture conditions designed to enrich for and proliferate the VM-CSC. During the processing of the tumor specimen, normal cells such as lymphocytes, stromal cells and connective tissue are eliminated. Upon completion of the expansion and purification steps, the enriched proliferating VM-CSC (tumor cells, TC) are inactivated by irradiation and placed in vapor phase liquid nitrogen storage. This process can take up to eight weeks, depending on the quantity and quality of the tumor specimen.

Once the tumor cell product has cleared quality assurance, the patient is notified to undergo a procedure called leukapheresis (usually a six liter procedure). This process entails the filtering of blood to collect peripheral blood mononuclear cells (PBMCs). The collected PBMC are shipped to the manufacturing facility by overnight courier for further purification by counter flow density centrifugation called elutriation. Elutriation is a process by which monocytes are purified from other lymphocytes in order to enrich for cells that can be turned into antigen presenting cells or dendritic cells. To generate the dendritic cells, the elutriated monocytes are incubated with the cytokines GM-CS F and interleukin-4 (IL-4) for six days.

On Day 6, the purified tumor cell product is removed from cryostorage, thawed and combined with the dendritic cells for 18-24 hours. This process results in “antigen loading” of the DC. The final product is either entirely DC or may contain some residual irradiated TC (which is considered permissible), and is referred to as DC-TC. The combined dendritic cell/tumor cell mixture is collected, cryopreserved to retain viability of the dendritic cells and stored in vapor phase liquid nitrogen.

Upon completion of the quality controls assays and release of the autologous cell therapy product, the batch is shipped to the treatment facility under vapor phase liquid nitrogen conditions. After arrival, the cell therapy product is stored under vapor phase liquid nitrogen conditions until prepared for administration.

Unless otherwise indicated, all numbers expressing quantities of ingredients, properties such as molecular weight, reaction conditions, and so forth used in the specification and claims are to be understood as being modified in all instances by the term “about.” As used herein the terms “about” and “approximately” means within 10 to 15%, preferably within 5 to 10%. Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

Thus, while there have shown and described and pointed out fundamental novel features of the disclosure as applied to an exemplary implementation and/or aspects thereof, it will be understood that various omissions, reconfigurations and substitutions and changes in the form and details of the exemplary implementations, disclosure and aspects thereof may be made by those skilled in the art without departing from the spirit of the disclosure and/or claims. For example, it is expressly intended that all combinations of those elements and/or method steps which perform substantially the same function in substantially the same way to achieve the same results are within the scope of the disclosure. Moreover, it should be recognized that structures and/or elements and/or method steps shown and/or described in connection with any disclosed form or implementation may be incorporated in any other disclosed or described or suggested form or implementation as a general matter of design choice. It is the intention, therefore, to not limit the scope of the disclosure. All such modifications are intended to be within the scope of the claims appended hereto.

All publications, patents, patent applications, references, and sequence listings, cited in this specification are herein incorporated by this reference as if fully set forth herein.

The Abstract is provided to comply with 37 CFR §1.72(b) to allow the reader to quickly ascertain the nature and gist of the technical disclosure. The Abstract is submitted with the understanding that it will not be used to interpret or limit the scope or meaning of the claims.

1. An immunogenic composition comprising dendritic cells activated ex vivo by tumor antigens derived from a population of purified vascular mimicry (VM) cancer stem cells (VM-CSCs).

2. The immunogenic composition of claim 1, wherein the tumor antigens comprise cell extracts of the VM-CSCs.

3. The immunogenic composition of claim 1, wherein the tumor antigens comprise lysates of the VM-CSCs.

4. The immunogenic composition of claim 1, wherein the tumor antigens comprise intact VM-CSCs.

5. The immunogenic composition of claim 4, wherein the intact VM-CSCs are rendered non-proliferative.

6. The immunogenic composition of claim 5 wherein the intact VM-CSCs are rendered non-proliferative by irradiation.

7. The immunogenic composition of claim 5, wherein the intact VM-CSCs are rendered non-proliferative by exposure of the cells to a nuclear cross-linking agent.

8. The immunogenic composition of claim 1, further comprising a pharmaceutically acceptable carrier or excipient.

9. The immunogenic composition of claim 1, further comprising an adjuvant.

10. The immunogenic composition of claim 9, wherein the adjuvant is granulocyte macrophage colony stimulating factor.

11. The immunogenic composition of claim 1, wherein the composition comprises activated dendritic cells and VM-CSCs.

12. A method of treating a cancer in a subject in need thereof, comprising administering an immunogenic dose of an immunogenic composition comprising dendritic cells activated ex vivo by tumor antigens derived from a population of purified VM-CSCs to the subject.

13. The method of claim 12 wherein the cancer is adrenocortical carcinoma, anal cancer, appendix cancer, astrocytoma, basal-cell carcinoma, bile duct cancer, bladder cancer, bone cancer, brain cancer, breast cancer, bronchial adenomas/carcinoids, carcinoid tumor, cervical cancer, chronic myeloproliferative disorders, colon cancer, desmoplastic small round cell tumor, endometrial cancer, ependymoma, esophageal cancer, Ewing's sarcoma, germ cell tumors, eye cancer, gallbladder cancer, gastric cancer, gastrointestinal carcinoid tumor, gastrointestinal stromal tumor (GIST), gestational trophoblastic tumor, glioma, gastric carcinoid, head and neck cancer, heart cancer, hepatocellular cancer, Hodgkin lymphoma, hypopharyngeal cancer, islet cell carcinoma, Kaposi sarcoma, kidney cancer, a leukemia, lip and oral cavity cancer, liposarcoma, liver cancer, lung cancer, a lymphoma, macroglobulinemia, medulloblastoma, melanoma, merkel cell carcinoma, mesothelioma, mouth cancer, multiple myeloma/plasma cell neoplasm, mycosis fungoides, nasal cavity and paranasal sinus cancer, nasopharyngeal carcinoma, neuroblastoma, oral cancer, oropharyngeal cancer, ovarian cancer, ovarian epithelial cancer, pancreatic cancer, parathyroid cancer, penile cancer, pharyngeal cancer, pheochromocytoma, pineal astrocytoma, pineal germinoma, pineoblastoma, pituitary adenoma, pleuropulmonary blastoma, prostate cancer, rectal cancer, renal cell carcinoma, retinoblastoma, rhabdomyosarcoma, salivary gland cancer, sarcoma, Sézary syndrome, skin cancer, squamous cell carcinoma, stomach cancer, testicular cancer, throat cancer, thymoma, thyroid cancer, urethral cancer, uterine cancer, uterine sarcoma, vaginal cancer, vulvar cancer, Waldenström macroglobulinemia, or Wilms tumor.

14. The method of claim 12, wherein the immunogenic composition is administered in a plurality of doses, each dose comprising about 5-20×10⁶ cells.

15. The method of claim 14, wherein the dose comprises about 10×10⁶ cells.

16. The method of claim 14, wherein the dose is administered weekly for 2-5 doses, followed by monthly for 3-6 doses.

17. The method of claim 14, wherein the subject receives from 6-10 doses of the immunogenic composition.

18. (canceled)

19. (canceled)

20. A method for preparing a population of VM-CSCs, the method comprising:
acquiring a sample of a tumor comprising tumor cells;
dissociating the tumor cells of the sample to form dissociated cells,
in vitro culturing the dissociated cells in a defined medium on a non-adherent substrate, wherein the defined medium is serum free and is supplemented with at least one growth factor that acts through the mitogen activated protein kinase (MAPK) pathway, thereby forming a population of vascular mimicry-cancer stem cell (VM-CSC) spheroids;
optionally in vitro culturing the CSC-spheroids to form early CSCs, mixed CSCs, or epithelial to mesenchymal transitioned (EMT)-CSCs; and
culturing the CSC spheroids, the early CSCs, mixed CSCs, or EMT-CSCs in a defined medium on an adherent substrate, wherein the defined medium contains a serum source and is supplemented with a soluble laminin, thereby forming a population of VM-CSCs, the VM-CSC population being characterized by at least 80% of the cells in the VM-CSC population expressing two or more of the biomarkers VEGF-R2, VE-cadherin, VEGF-A, CD34, vWF, and PECAM.

21. The method of claim 20, wherein the defined medium of any of the steps further comprises at least one receptor tyrosine kinase (RTK) ligand.

22. The method of claim 20, wherein the soluble laminin comprises an alpha1, alpha2, alpha3, or alpha4 chain.

23. The method of claim 20, wherein the laminin is in a monomer, dimer, or trimer form.

24. The method of claim 20, wherein the laminin is not an insoluble polymer form.

25. The method of claim 20, the VM-CSC population being characterized by at least 80% of the cells in the VM-CSC population further expressing one or more of the biomarkers VEGF-R1 and UEA-1.

26. The method of claim 20, the VM-CSC population being characterized by at least 90% of the cells in the VM-CSC population expressing two or more of the biomarkers VEGF-R2, VE-cadherin, VEGF-A, CD34, vWF, and PECAM.

27. The method of claim 20, the CSC spheroid population being characterized by at least 80% of the cells in the CSC spheroid population expressing two or more of the biomarkers EpCAM, CD117, ALDH, CD133, CD24, Ki-67.

28. The method of claim 20, the CSC spheroid population being characterized by at least 80% of the cells in the CSC spheroid population further expressing one or more of the biomarkers NCAM, vimentin, CK8, TGFβR, EGFR, CD44, ABCG2, Slug/Snail, nestin, and TP53.

29. The method of claim 20, the CSC spheroid population being characterized by at least 90% of the cells in the CSC spheroid population expressing two or more of the biomarkers EpCAM, CD117, ALDH, CD133, CD24, Ki-67.

30. The method of claim 20, comprising generating the early CSC by:
culturing the CSC spheroids in a defined medium on an adherent substrate, wherein the defined medium is serum free and is supplemented with at least one growth factor that acts through the MAPK pathway, thereby forming a population of early CSCs, the population of early CSCs being characterized by at least 80% of the cells in the early CSC population expressing two or more of the biomarkers EpCAM, CD133, CD44, Nanog, Sox2, Oct3/4, CD17, and Ki-67.

31. The method of claim 30, the early CSC population being characterized by at least 80% of the cells in the early CSC population further expressing one or more of the biomarkers TGFβR and CD24.

32. The method of claim 30, the early CSC population being characterized by at least 90% of the cells in the early CSC population expressing two or more of the biomarkers EpCAM, CD133, CD44, Nanog, Sox2, Oct3/4, CD17, and Ki-67.

33. The method of claim 20, comprising generating the mixed CSCs by:
culturing the CSC spheroids in a defined medium on an adherent substrate, wherein the defined medium contains serum, thereby forming a population of mixed CSCs, the population of mixed CSCs being characterized by at least 80% of the cells in the mixed CSC population expressing two or more of the biomarkers ABCG2, CD133, CD24, CD44, CD34, CD117, CK8, EpCAM, Ki-67, Nanog, N-cadherin, NCAM, Oct3/4, Slug/Snail, Twist, vimentin, ALDH, TGFβR, Sox2, EGFR) nestin, TP53, VEGF-R1, VEGF-R2, VE-cadherin, VEGF-A, vWF, PECAM, and UEA-1.

34. The method of claim 33, wherein the defined medium further comprises at least one receptor tyrosine kinase (RTK) ligand.

35. The method of claim 33, the mixed CSC population being characterized by at least 90% of the cells in the mixed CSC population expressing two or more of the biomarkers ABCG2, CD133, CD24, CD44, CD34, CD117, CK8, EpCAM, Ki-67, Nanog, N-cadherin, NCAM, Oct3/4, Slug/Snail, Twist, vimentin, ALDH, TGFβR, Sox2, EGFR) nestin, TP53, VEGF-R1, VEGF-R2, VE-cadherin, VEGF-A, vWF, PECAM, and UEA-1.

36. The method of claim 20, comprising generating the EMT-CSCs by:
culturing the CSC spheroids in a defined medium on an adherent substrate, wherein the defined medium contains serum and is supplemented with at least one growth factor that acts through the MAPK pathway, thereby forming a population of EMT-CSCs, the population of EMT-CSCs being characterized by at least 80% of the cells in the EMT-CSC population expressing two or more of the biomarkers NCAM, Slug/Snail, CD24, and Twist.

37. The method of claim 36, the population of EMT-CSCs being characterized by at least 80% of the cells in the EMT-CSC population further expressing one or more of the biomarkers CD133, Nanog, CD117, N-cadherin, CD44, and vimentin.

38. The method of claim 36, the population of EMT-CSCs being characterized by at least 90% of the cells in the EMT-CSC population expressing two or more of the biomarkers NCAM, Slug/Snail, CD24, and Twist.

39. A method for preparing a population of VM-CSCs, the method comprising:
acquiring a sample of a tumor comprising tumor cells;
dissociating the tumor cells of the sample to form dissociated cells;
in vitro culturing the dissociated cells in a defined medium on a non-adherent substrate, wherein the defined medium is serum free and is supplemented with at least one growth factor that acts through the MAPK pathway, thereby forming CSC spheroids;
in vitro culturing the CSC spheroids to form early CSCs, mixed CSCs, or EMT-CSCs; and
in vitro culturing the early CSCs, mixed CSCs, or EMT-CSCs in a defined medium on an adherent substrate, wherein the defined medium is supplemented with a soluble laminin, thereby forming a population of VM-CSCs;
the population of VM-CSCs being characterized by the at least 80% of the cells in the VM-CSC population expressing two or more of the biomarkers VEGF-R2, VE-cadherin, VEGF-A, CD34, vWF, and PECAM.

40. The method of claim 39, wherein the defined medium in any of the steps further comprises at least one RTK ligand.

41. The method of claim 39, the population of VM-CSCs being characterized by at least 80% of the cells in the VM-CSC population further expressing one or more of the biomarkers VEGF-R1 and UEA-1.

42. The method of claim 39, the population of VM-CSCs being characterized by at least 90% of the cells in the VM-CSC population expressing two or more of the biomarkers VEGF-R2, VE-cadherin, VEGF-A, CD34, vWF, and PECAM.

43. The method of claim 39, wherein the soluble laminin comprises an alpha1, alpha2, alpha3, or alpha4 chain.

44. The method of claim 39, wherein the laminin is in a monomer, dimer, or trimer form.

45. The method of claim 39, wherein the laminin is not an insoluble polymer form.

46. The method of claim 20, wherein the defined media is any media described in Table 2.

47. The method of claim 20, wherein the defined media is any media from a combination of Table 2 and Table 3.

48. The method of claim 20, wherein the defined media is any media from a combination of Table 2, Table 3, and Table 4.

49. The method of claim 20, wherein the defined media is any media from a combination of Table 2 and Table 4.

50. The method of claim 39, wherein the growth factor is one or more of fibroblast growth factor (FGF), epidermal growth factor (EGF), or activin A.

51. The method of claim 50, wherein the FGF is basic FGF (bFGF).

52. The method of claim 20, wherein the defined medium is not supplemented with activin A.

53. The method of claim 20, wherein the defined medium is supplemented with an antagonist of activin A, in an amount effective to prevent spontaneous differentiation of CSCs.

54. The method of claim 20, wherein the media is supplemented with an antagonist of activin A, and the antagonist is follistatin or an antibody that specifically binds to activin A.

55. The method of claim 20, wherein the medium is not supplemented with an antioxidant.

56. The method of claim 55, wherein the antioxidant is superoxide dismutase, catalase, glutathione, putrescine, or β-mercaptoethanol.

57. The method of claim 20, wherein the medium is supplemented with glutathione.

58. The method of claim 20, wherein the adherent substrate is configured to adhere to, and to collect, anchorage dependent cells.

59. The method of claim 58, wherein the anchorage dependent cells are fibroblasts.

60. The method of claim 20, wherein the non-adherent substrate is an ultralow adherent polystyrene surface.

61. The method of claim 20, wherein the adherent substrate comprises a surface coated with a protein rich in RGD tripeptide motifs.

62. A population of purified VM-CSCs prepared by the method of claim 20.

63. A VM-CSC cell line prepared by the method of claim 20.

64. A method of stimulating an immune response against antigens of a tumor comprising vascular mimicry cancer cells in a subject in need thereof, comprising administering an immunogenic dose of the immunogenic composition of claim 1 to the subject.

65. (canceled)

66. (canceled)

67. The method of claim 39, wherein the defined media is any media described in Table 2.

68. The method of claim 39, wherein the defined media is any media from a combination of Table 2 and Table 3.

69. The method of claim 39, wherein the defined media is any media from a combination of Table 2, Table 3, and Table 4.

70. The method of claim 39, wherein the defined media is any media from a combination of Table 2 and Table 4.

71. The method of claim 39, wherein the defined medium is not supplemented with activin A.

72. The method of claim 39, wherein the defined medium is supplemented with an antagonist of activin A, in an amount effective to prevent spontaneous differentiation of CSCs.

73. The method of claim 39, wherein the media is supplemented with an antagonist of activin A, and the antagonist is follistatin or an antibody that specifically binds to activin A.

74. The method of claim 39, wherein the medium is not supplemented with an antioxidant.

75. The method of claim 74, wherein the antioxidant is superoxide dismutase, catalase, glutathione, putrescine, or β-mercaptoethanol.

76. The method of claim 39, wherein the medium is supplemented with glutathione.

77. The method of claim 39, wherein the adherent substrate is configured to adhere to, and to collect, anchorage dependent cells.

78. The method of claim 77, wherein the anchorage dependent cells are fibroblasts.

79. The method of claim 39, wherein the non-adherent substrate is an ultralow adherent polystyrene surface.

80. The method of claim 39, wherein the adherent substrate comprises a surface coated with a protein rich in RGD tripeptide motifs.

81. A population of purified VM-CSCs prepared by the method of claim 39.

82. A VM-CSC cell line prepared by the method of claim 39.

83. A method of stimulating an immune response against antigens of a tumor comprising vascular mimicry cancer cells in a subject in need thereof, comprising administering an immunogenic dose of the VM-CSCs of claim 62 to the subject.

84. A method of stimulating an immune response against antigens of a tumor comprising vascular mimicry cancer cells in a subject in need thereof, comprising administering an immunogenic dose of the VM-CSC cell line of claim 63 to the subject.

85. A method of stimulating an immune response against antigens of a tumor comprising vascular mimicry cancer cells in a subject in need thereof, comprising administering an immunogenic dose of the VM-CSCs of claim 81 to the subject.

86. A method of stimulating an immune response against antigens of a tumor comprising vascular mimicry cancer cells in a subject in need thereof, comprising administering an immunogenic dose of the VM-CSC cell line of claim 82 to the subject.
